(19) United States
(12) Patent Application Publication
(10) Pub. No.: US 2016/0151482 A1
(43) Pub. Date: Jun. 2, 2016

(54) MESOPOROUS ALUM NANOPARTICLES AS A UNIVERSAL PLATFORM FOR ANTIGEN ADSORPTION, PRESENTATION, AND DELIVERY

(71) Applicants: STC. UNM, Albuquerque, NM (US); SANDIA CORPORATION, Albuquerque, NM (US)

(72) Inventors: Eric C. Carnes, Albuquerque, NM (US); C. Jeffrey Brinker, Albuquerque, NM (US); Carlee Erin Ashley, Albuquerque, NM (US)

(21) Appl. No.: 14/781,765

(22) PCT Filed: Apr. 2, 2014
(86) PCT No.: PCT/US14/32711

§ 371 Date: Nov. 23, 2015

Related U.S. Application Data
(60) Provisional application No. 61/807,706, filed on Apr. 2, 2013.

(51) Int. Cl. A61K 39/39; A61K 9/14; A61K 9/51

(52) U.S. Cl. CPC A61K 39/39; A61K 9/5115; A61K 9/146; A61K 2039/55505

(57) ABSTRACT

The present invention relates to mesoporous alum nanoparticles which can be used as a universal platform for antigen adsorption, presentation and delivery to provide immune compositions, including vaccines and to generate an immune response (preferably, both humoral and cell mediated immune response), preferably a heightened immune response to the presentation of one or more antigens to a patient or subject.

### CROSS REFERENCE TO RELATED APPLICATIONS

This application claims priority from U.S. Provisional Patent Application No. 61/807,706, entitled “Mesoporous Alum Nanoparticles as a Universal Platform for Antigen Adsorption, Presentation, and Delivery”, filed Apr. 2, 2013. The complete contents of this provisional application are hereby incorporated by reference in their entirety.

### STATEMENT REGARDING FEDERAL FUNDING

This invention was developed under Contract DE-AC04-94AL85000 between Sandia Corporation and the U.S. Department of Energy. Accordingly, the United States has certain rights in this invention.

### FIELD OF THE INVENTION

The present invention relates to mesoporous nanoparticles, such as mesoporous alum nanoparticles (MANPs) and mesoporous silica nanoparticles (MSNPs) which can be used as a universal platform for antigen adsorption, presentation and delivery to provide immune compositions, including vaccines and to generate an immune response (preferably, both humoral and cell mediated immunoe responses), preferably a heightened immune response to the presentation of one or more antigens to a patient or subject.

In certain preferred embodiments, the invention provides protocells comprising a porous alum based nanoparticle which is surrounded by a supported lipid or polymer bilayer or multilayer, preferably a supported lipid bilayer (SLB).

### BACKGROUND OF THE INVENTION

Aluminum salts, including aluminum hydroxide, aluminum phosphate, and potassium aluminum sulfate (also known as ‘alum’) have been approved for use as adjuvants for over six decades and are effective at stimulating T-helper 2 (Th2 or humoral) immunity.¹ Although the mechanism of action of aluminum-based adjuvants remains unclear, it has been postulated that they act as a depot for antigen at the injection site and, due to their particulate nature, trigger efficient uptake of antigen by APCs.¹

More recently, alum has been shown to activate the NALP3 inflammasome in a Toll-like receptor (TLR)-independent fashion, which leads to secretion of mature IL-1-family cytokines (e.g. IL-1β) by peripheral blood and bone marrow-derived mononuclear cells.^{2, 3} Despite their widespread use, however, aluminum salt adjuvants have several limitations, including ineffectiveness for some antigens, injection site reactions, especially upon subcutaneous or intradermal administration, and stimulation of eosinophilia and IgE production, which increase the risk of vaccine allergy or anaphylaxis.⁴ Alum, furthermore, fails to induce CD8⁺ T (CD8T) cell responses, which are especially critical for effective vaccination against intracellular pathogens.⁵

Accordingly, the need exists for therapeutic MANPs which can be made by commercially practicable processes and which provide targeted delivery of active ingredients that are effective in the treatment of a wide variety of pathogens.

### SUMMARY OF THE INVENTION

The inventors provide mesoporous alum nanoparticles (MANPs), including high-surface-area MANPs, having pore sizes and surface chemistries that facilitate facile adsorption and presentation of antigens isolated from several Category A and B biothreat agents.

More specifically, in various embodiments, our novel mesoporous alum nanoparticles are characterized by any one or more or all of the following properties: (1) comprise about 50% to about 70% by weight of a therapeutic antigen cargo (2) have a pore size of less than about 1 nm (in some instances about 0.03 nm, but often at least about 1 nm) to approximately 75 nm (3) have a surface area of approximately 75 m²/g to approximately 1,500 m²/g and a diameter of approximately 50 nm to 50 μm (4) are made by aerosol-assisted evaporation-induced self-assembly (5) deliver antigen cargo in a pH-dependent manner (6) uniquely target antigen-presenting cells (APCs), and (7) are readily encapsulated by a wide variety of lipids to yield therapeutically effective protocells.

In one embodiment, in order to induce optimal humoral and cellular immune responses, MANPs are loaded with cocktails of antigens and, if necessary, immunostimulatory (immunogenic) molecule(s) and are encapsulated within a supported lipid bilayer (SLB). The encapsulated MANPs can be further modified with targeting ligands that promote uptake by APCs and cytosolic release of encapsulated antigen(s). Targeting ligands are exemplified in Example 3.

Our novel application of aerosol-assisted evaporation-induced self-assembly provides MANPs which are mesoporous, which can be stably loaded with high concentrations of various antigens (preferably protein antigens but including in certain embodiments carbohydrate antigens (containing a carbohydrate mimotope), lipoproteins or glycoproteins and which may be engineered for burst or sustained release profiles. Aerosol-assisted evaporation-induced self-assembly enables modification of a nanoparticle surface with various targeting ligands and promotes effective uptake by antigen-presenting cells. Further, antigen-loaded mesoporous oxide nanoparticles induce antigen-specific humoral and cellular immune responses.

Accordingly, in certain aspects, the present invention is directed to a cell-targeting mesoporous alum nanoparticle comprising a nanoporous alum with an optional supported lipid bilayer; at least one antigen and optionally at least one immunostimulatory (immunogenic) molecule (which also may be expressed by plasmid DNA); and at least one further component selected from the group consisting of a cell targeting species and/or a ligand that facilitates uptake of the nanoparticles by antigen-presenting cells (APCs) and/or cytosolic dispersion of antigen (targeting ligand);
a fusogenic peptide that promotes endosomal escape of nanoparticles and encapsulated DNA, and other cargo comprising at least one additional cargo component (other than the antigen) selected from the group consisting of polynucleotides (DNA or RNA), including double stranded linear DNA, minicircle DNA or a plasmid DNA (including plasmid DNA which is capable of expressing an immunostimulatory (immunogenic) molecule as otherwise described herein;

at least one drug;

an imaging agent, RNA, including mRNA, small interfering RNA, small hairpin RNA, microRNA, immunostimulatory RNA (isRNA) or a mixture thereof, wherein one of said cargo components is optionally conjugated further with a nuclear localization sequence.

Pharmaceutical compositions comprising a plurality of MANPs as described herein, and, optionally, a pharmaceutically-acceptable excipient, are also provided.

These and other aspects of the invention are described further in the Detailed Description of the Invention.

### BRIEF DESCRIPTION OF THE FIGURES

FIG. 1. Gallery of mesoporous oxide nanoparticles prepared by aerosol-assisted EISA with hexagonal (A), cubic (B), lamellar (C), and cellular (D-E) pore geometries. (F) shows dual-templated particles with interconnected 2-nm and 60-nm pores. As determined in the experiment(s) of Example 1.

FIG. 2. FIG. 2 illustrates that MSNPs have a high capacity for physicochemically disparate proteins and maintain long-term stability of encapsulated proteins in the absence of cold chain. As determined in the experiment(s) of Example 2.

FIG. 3. FIG. 3 illustrates the degree of condensation of the MSNP framework can be optimized for burst or sustained release of encapsulated OVA. As determined in the experiment(s) of Example 2.

FIG. 4. FIG. 4 illustrates the encapsulation of OVA-loaded MSNPs in a SLB that is further modified with targeting ligands enables efficient uptake by dendritic cells and macrophages and pH-triggered release of OVA. As determined in the experiment(s) of Example 3.

FIG. 5. FIG. 5 illustrates the in vitro and in vivo assessment of MPLA-targeted, OVA-loaded MSNPs in the absence and presence of isRNA. As determined in the experiment(s) of Example 4.

FIG. 6. FIG. 6 illustrates a schematic of a MANP for antigen adsorption, presentation, and delivery.

FIG. 7. FIG. 7 shows a schematic perspective side view of an embodiment of a protocell embodiment of the invention.

### DETAILED DESCRIPTION OF THE INVENTION

The following terms shall be used throughout the specification to describe the present invention. Where a term is not specifically defined herein, that term shall be understood to be used in a manner consistent with its use by those of ordinary skill in the art.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention. In instances where a substituent is a possibility in one or more Markush groups, it is understood that only those substituents which form stable bonds are to be used.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

Furthermore, the following terms shall have the definitions set out below.

The term “patient” or “subject” is used throughout the specification within context to describe an animal, generally a mammal, especially including a domesticated animal (e.g. dog, cat, cow, horse, pig, sheep, goat, among others) and preferably a human, to whom treatment, including especially prophylactic treatment (prophylaxis), with the compositions according to the present invention is provided. For treatment of those infections, conditions or disease states which are specific for a specific animal such as a human patient, the term patient refers to that specific animal. In most instances, the patient or subject of the present invention is a human patient of either or both genders.

The term “effective” is used herein, unless otherwise indicated, to describe an amount of a compound or component which, when used within the context of its use, produces or effects an intended result, whether that result relates to the prophylaxis and/or therapy of an infection and/or disease state or as otherwise described herein. The term effective subsumes all other effective amount or effective concentration terms (including the term “therapeutically effective”) which are otherwise described or used in the present application.

The term “compound” is used herein to describe any specific compound or bioactive agent disclosed herein, including any and all stereoisomers (including diasteromers), individual optical isomers (enantiomers), mixtures of stereoisomers in any ratio, including racemic mixtures, isotopologues, pharmaceutically acceptable salts and prodrug forms. The term compound herein refers to stable compounds. Within its use in context, the term compound may refer to a single compound or a mixture of compounds as otherwise described herein.

The term “bioactive agent” refers to any biologically active compound or drug which may be formulated for use in an embodiment of the present invention. Exemplary bioactive agents include the compounds according to the present invention which are used to treat microbial infections, including bacteria and viruses as otherwise described herein.

The terms “treat”, “treating”, and “treatment”, are used synonymously to refer to any action providing a benefit to a patient at risk for or afflicted with a disease, including improvement in the condition through lessening, inhibition, suppression or elimination of at least one symptom, delay in progression of the disease, prevention, delay in or inhibition of the likelihood of the onset of the disease, etc. In the case of viral infections, these terms also apply to viral infections and preferably include, in certain particularly favorable embodiments the eradication or elimination (as provided by limits of diagnostics) of the virus which is the causative agent of the infection.

Treatment, as used herein, encompasses both prophylactic and therapeutic treatment, but especially prophylactic treatment. Compositions according to the present invention can, for example, be administered prophylactically to a mammal in advance of the occurrence of disease to reduce the likelihood of that disease. Prophylactic administration is effective to reduce or decrease the likelihood of the subsequent occurrence of disease in the mammal, or decrease the severity of disease (inhibition) that subsequently occurs. Alternatively, compounds according to the present invention can, for example, be administered therapeutically to a mammal that is already afflicted by disease. Administration of the compositions according to the present invention is effective to decrease the severity of the disease or lengthen the lifespan of the mammal so afflicted, as in the case of a microbial infection or cancer, or inhibit or even eliminate the causative agent of the disease.

The term “coadministration” as used herein to describe the administration of a composition according to the present invention which comprises nanoparticles as otherwise described herein, in combination with at least one additional agent, such as an immunostimulatory (immunogenic) molecule as otherwise described herein or another biologically active agent, in effective amounts. Although the term coadministration preferably includes the administration of two or more compositions and/or active agents to the patient at the same time, it is not necessary that the compositions actually be administered at the exact same time, only that amounts of composition and/or compound will be administered to a patient or subject such that effective concentrations are found in the blood, serum or plasma, or in the pulmonary tissue within a patient or subject at the same time.

The term “pharmaceutically acceptable” as used herein means that the compound or composition is suitable for administration to a subject, including a human patient, to achieve the treatments described herein, without unduly deleterious side effects in light of the severity of the disease and necessity of the treatment.

The term “inhibit” as used herein refers to the partial or complete elimination of a potential effect, while inhibitors are compounds/compositions that have the ability to inhibit.

The term “prevention” when used in context shall mean “reducing the likelihood” or preventing a disease, condition or disease state from occurring as a consequence of administration or concurrent administration of one or more compounds or compositions according to the present invention, alone or in combination with another agent. It is noted that prevention will rarely be 100% effective; consequently the terms prevention and reducing the likelihood are used to denote the fact that within a given population of patients or subjects, administration with compounds according to the present invention will reduce the likelihood or inhibit a particular condition or disease state (in particular, the worsening of a disease state such as the growth or metastasis of cancer) or other accepted indicators of disease progression from occurring.

The term “nanoparticle” is used to describe a porous nanoparticle which is made of a material comprising alum or silica as otherwise defined herein. In certain aspects, a porous alum based nanoparticle is used for the preferred protocells and is surrounded by a supported lipid or polymer bilayer or multilayer, preferably a supported lipid bilayer (SLB). Various embodiments according to the present invention provide nanostructures and methods for constructing and using the nanostructures and providing nanoparticles according to the present invention. Porous/mesoporous alum particles of varying sizes ranging in size (diameter) from less than 5 nm to 200 nm or 500 nm or more are readily available in the art or can be readily prepared using methods known in the art (see the examples section in attached Appendix A). Nanoparticles used in the present invention may be readily obtained using methodologies known in the art. The examples section of the present application in attached Appendix A provides certain methodology for obtaining protocells which are useful in the present invention. Nanoparticles according to the present invention may be readily prepared, including nanoparticles comprising lipids which are fused to the surface of the nanoparticle. See, the examples in the attached Appendix A or by analogy from for example, Liu, et al., Chem. Comm., 5100-5102 (2009), Liu, et al., J. Amer. Chem. Soc., 131, 1354-1355 (2009), Liu, et al., J. Amer. Chem. Soc., 131, 7567-7569 (2009) Lu, et al., Nature, 398, 223-226 (1999). Preferred MANPS for use in the present invention are prepared according to the procedures which are described in the experimental section which follows.

In an embodiment of the present invention, the nanostructures include a core-shell structure which comprises a porous particle core surrounded by a shell of lipid preferably a bilayer, but possibly a monolayer or multilayer. The porous particle core can include, for example, a porous nanoparticle made of an inorganic and/or organic material as set forth above surrounded by a lipid bilayer. In the present invention, these lipid bilayer surrounded nanostructures are referred to as “protocells” or “functional protocells,” since they have a supported lipid bilayer membrane structure. In embodiments according to the present invention, the porous particle core of the protocells can be loaded with various desired species (“cargo”), especially including antigens, small molecules (e.g. bioactive agents as otherwise described herein), large molecules (e.g. including macromolecules such as RNA, including small interfering RNA or siRNA or small hairpin RNA or shRNA. In certain aspects of the invention, the MANPS are loaded with antigen and optionally, super-coiled plasmid DNA, which can be used to deliver the antigenic peptide(s) or a small hairpin RNA/shRNA or small interfering RNA/siRNA.

In certain embodiments, the cargo components can include, but are not limited to, chemical small molecules (especially antibiotics and antiviral agents). In certain embodiments, the lipid bilayer of the nanoparticles can provide biocompatibility and can be modified to possess targeting species including, for example, targeting peptides including antibodies, aptamers, and PEG (polyethylene glycol) to allow, for example, further stability of the nanoparticles and/or a targeted delivery into a bioactive cell.

The MANPS particle size distribution, depending on a given application, may be monodisperse or polydisperse. The particle cores may be monodisperse (i.e., a uniform sized population varying no more than about 5% in diameter e.g., ±10-nm for a 200 nm diameter protocell prepared using solution techniques) or polydisperse (i.e., a polydisperse population can vary widely from a mean or medium diameter, e.g., up to ±200-nm or more if prepared by aerosol). Polydisperse populations can be sized into monodisperse populations. All of these are suitable for nanoparticle formation. In the present invention, preferred nanoparticles are preferably no more than about 500 nm in diameter, preferably no more than about 200 nm in diameter (preferably about 2 nm to about 50 nm) in order to afford delivery to a patient or subject and produce an intended immune effect.

Nanoparticles according to the present invention generally range in size from about 2 nm to greater than about 50 nm, about 2 to about 500 nm, about 8-10 nm up to about 5 μm in diameter, preferably about 20-nm-3 μm in diameter, about 10 nm to about 100 nm, more preferably about 5-50 nm. As discussed above, the MANPS population may be considered monodisperse or polydisperse based upon the mean or median diameter of the population of protocells. Size is very important to immune aspects of the present invention as particles smaller than about 8-nm diameter are excreted through kidneys, and those particles larger than about 200 nm are trapped by the liver and spleen. Thus, an embodiment of the present invention focuses in smaller sized protocells (preferably, about 2 nm to about 50 nm) for drug delivery and diagnostics in the patient or subject.

Nanoparticles are characterized by mesopores that may intersect the surface of the nanoparticle (by having one or both ends of the pore appearing on the surface of the nanoparticle) or that may be internal to the nanostructure with at least one or more mesopore interconnecting with the surface mesopores of the nanoparticle. Interconnecting pores of smaller size are often found internal to the surface mesopores. The overall range of pore size can be 0.03-50-nm in diameter. Preferred pore sizes of mesopores range from about 2-30 nm (preferably about 2 to about 20 nm); they can be monosized or bimodal or graded—they can be ordered or disordered (essentially randomly disposed or worm-like).

Mesopores (IUPAC definition 2-50-nm in diameter) are ‘molded’ by templating agents including surfactants, block copolymers, molecules, macromolecules, emulsions, latex beads, or nanoparticles. In addition, processes could also lead to micropores (IUPAC definition less than 2-nm in diameter) all the way down to about 0.03-nm e.g. if a templating moiety in the aerosol process is not used. They could also be enlarged to macropores, i.e., equal to or greater than 50-nm in diameter.

Pore surface chemistry of the nanoparticle material can be very diverse—pore surface chemistry, especially charge and hydrophobicity, affect loading capacity. Attractive electrostatic interactions or hydrophobic interactions control/enhance loading capacity and control release rates. Higher surface areas can lead to higher loadings of drugs/cargos through these attractive interactions.

The surface area of nanoparticles, as measured by the N2 BET method, ranges from about 100 m²/g to >about 1,200 m²/g. In general, the larger the pore size, the smaller the surface area. The surface area theoretically could be reduced to essentially zero, if one does not remove the templating agent or if the pores are sub-0.5-nm and therefore not measurable by N2 sorption at 77K due to kinetic effects. However, in this case, they could be measured by CO2 or water sorption, but would probably be considered non-porous. This would apply if biomolecules are encapsulated directly in the silica cores prepared without templates, in which case particles (internal cargo) would be released by dissolution of the silica matrix after delivery to the cell.

Typically the MANPS according to the present invention are loaded with cargo to a capacity up to about 50 weight %: defined as (cargo weight/weight of loaded protocell)×100. The optimal loading of cargo is often about 0.01 to 10% but this depends on the drug or drug combination which is incorporated as cargo into the MANPS. This is generally expressed in μM per 10¹⁰ particles where we have values ranging from 2000-100 μM per 10¹⁰ particles. Preferred MANPS according to the present invention exhibit release of cargo at pH about 5.5, which is that of the endosome, but are stable at physiological pH of 7 or higher (such as pH 7.4).

The surface area of the internal space for loading is the pore volume whose optimal value ranges from about 1.1 to 0.5 cubic centimeters per gram (cc/g). Note that in the MANPS according to one embodiment of the present invention, the surface area is mainly internal as opposed to the external geometric surface area of the nanoparticle.

The lipid bilayer supported on the porous particle according to one embodiment of the present invention has a lower melting transition temperature, i.e. is more fluid than a lipid bilayer supported on a non-porous support or the lipid bilayer in a liposome. This is sometimes important in achieving high affinity binding of immune peptides or targeting ligands at low peptide densities, as it is the bilayer fluidity that allows lateral diffusion and recruitment of peptides by target cell surface receptors. One embodiment provides for peptides to cluster, which facilitates binding to a complementary target.

In the present invention, the lipid bilayer may vary significantly in composition. Ordinarily, any lipid or polymer which may be used in liposomes may also be used in MANPS according to the present invention. Preferred lipids are as otherwise described herein.

The charge of the mesoporous MANPS NP core as measured by the Zeta potential may be varied monotonically from −50 to +50 mV and accordingly as described herein. This charge modification, in turn, varies the loading of the antigen and optional drug within the cargo of the protocell. Generally, after fusion of the supported lipid bilayer, the zeta-potential is reduced to between about −10 mV and +5 mV, which is important for maximizing circulation time in the blood and avoiding non-specific interactions.

Further characteristics of MANPS according to an embodiment of the present invention are that they are stable at pH 7, i.e. they don't leak their cargo, but at pH 5.5, which is that of the endosome, the lipid or polymer coating becomes destabilized, thus initiating cargo release. This pH-triggered release is important for maintaining stability of the MANPS up until the point that it is internalized in the cell by endocytosis, whereupon several pH triggered events cause release into the endosome and consequently, the cytosol of the cell.

The term “lipid” is used to describe the components which are used to form lipid bilayers on the surface of the nanoparticles which are used in the present invention. Various embodiments provide nanostructures which are constructed from nanoparticles which support a lipid bilayer(s). In embodiments according to the present invention, the nanostructures preferably include, for example, a core-shell structure including a porous particle core surrounded by a shell of lipid bilayer(s). The nanostructure, preferably a porous alum nanostructure as described above, supports the lipid bilayer membrane structure.

In embodiments according to the invention, the lipid bilayer of the protocells can provide biocompatibility and can be modified to possess targeting species including, for example, antigens, targeting peptides, fusogenic peptides, antibodies, aptamers, and PEG (polyethylene glycol) to allow, for example, further stability of the protocells and/or a targeted delivery into a cell to maximize an immune response. PEG, when included in lipid bilayers, can vary widely in molecular weight (although PEG ranging from about 10 to about 100 units of ethylene glycol, about 15 to about 50 units, about 15 to about 20 units, about 15 to about 25 units, about 16 to about 18 units, etc, may be used) and the PEG component which is generally conjugated to phospholipid through an amine group (often an ethanolamine group) comprises about 1% to about 20%, preferably about 5% to about 15%, about 10% by weight of the lipids which are included in the lipid bilayer.

Numerous lipids which are used in liposome delivery systems may be used to form the lipid bilayer on nanoparticles to provide MANPS according to the present invention. Virtually any lipid which is used to form a liposome may be used in the lipid bilayer which surrounds the nanoparticles to form MANPS according to an embodiment of the present invention. Preferred lipids for use in the present invention include, for example, 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof. Cholesterol, not technically a lipid, but presented as a lipid for purposes of an embodiment of the present invention given the fact that cholesterol may be an important component of the lipid bilayer of protocells according to an embodiment of the invention. Often cholesterol is incorporated into lipid bilayers of protocells in order to enhance structural integrity of the bilayer. These lipids are all readily available commercially from Avanti Polar Lipids, Inc. (Alabaster, Ala., USA). DOPE and DPPE are particularly useful for conjugating (through an appropriate crosslinker) peptides, polypeptides, including immune peptides, proteins and antibodies, RNA and DNA through the amine group on the lipid.

The term “immunostimulatory molecule” or “immunogenic molecule” is used to describe any molecule which may be added to compounds according to the present invention to stimulate an immune response in the patient or subject to which the present compositions are administered Immunostimulatory molecules (immunogenic molecules) for use in the present invention include a cytokine such as an interleukin, such as IL-2, IL-4, KL-5, IL-6, IL-7, IL-8, IL-9, IL-10, IL-12, IL-13, IL-15, for example or an interferon (IFN) such as IFN-α, IFN-β, IFN-γ, or a pegylated IFN, or GM-CSF and various other cytokines, a tumor necrosis factor, including TNF-alpha and TNF-beta, as well as molecules such as andrographolide, 14-deoxyandrographolide and 14-deoxy-11,12-didehydroandrographolide, among others. Mixtures of these immunostimulatory molecules are contemplated for use in the present invention. These molecules may be included as cargo in nanoparticles according to the present invention or alternatively, these molecules may include immune stimulatory RNA (isRNA) or be expressed by plasmid DNA as otherwise described herein which may be included in nanoparticles according to the present invention. Alternatively, one or more immunostimulatory molecules may be co-administered with compositions which comprise nanoparticles according to the present invention.

The term “reporter” is used to describe an imaging agent or moiety which is incorporated into the phospholipid bilayer or cargo of MANPS according to an embodiment of the present invention and provides a signal which can be measured. The moiety may provide a fluorescent signal or may be a radioisotope which allows radiation detection, among others. Exemplary fluorescent labels for use in protocells (preferably via conjugation or adsorption to the lipid bilayer or silica core, although these labels may also be incorporated into cargo elements such as DNA, RNA, polypeptides and small molecules which are delivered to cells by the protocells, include Hoechst 33342 (350/461), 4′,6-diamidino-2-phenylindole (DAPI, 356/451), Alexa Fluor® 405 carboxylic acid, succinimidyl ester (401/421), CellTracker™ Violet BMQC (415/516), CellTracker™ Green CMFDA (492/517), calcein (495/515), Alexa Fluor® 488 conjugate of annexin V (495/519), Alexa Fluor® 488 goat anti-mouse IgG (H+L) (495/519), Click-iT® AHA Alexa Fluor® 488 Protein Synthesis HCS Assay (495/519), LIVE/DEAD® Fixable Green Dead Cell Stain Kit (495/519), SYTOX® Green nucleic acid stain (504/523), MitoSOX™ Red mitochondrial superoxide indicator (510/580). Alexa Fluor® 532 carboxylic acid, succinimidyl ester (532/554), pHrodo™ succinimidyl ester (558/576), CellTracker™ Red CMTPX (577/602), Texas Red® 1,2-dihexadecanoyl-sn-glycero-3-phosphoethanolamine (Texas Red® DHPE, 583/608), Alexa Fluor® 647 hydrazide (649/666), Alexa Fluor® 647 carboxylic acid, succinimidyl ester (650/668), Ulysis™ Alexa Fluor® 647 Nucleic Acid Labeling Kit (650/670) and Alexa Fluor® 647 conjugate of annexin V (650/665). Moities which enhance the fluorescent signal or slow the fluorescent fading may also be incorporated and include SlowFade® Gold antifade reagent (with and without DAPI) and Image-iT® FX signal enhancer. All of these are well known in the art. Additional reporters include polypeptide reporters which may be expressed by plasmids (such as histone-packaged supercoiled DNA plasmids) and include polypeptide reporters such as fluorescent green protein and fluorescent red protein. Reporters pursuant to the present invention are utilized principally in diagnostic applications including diagnosing the existence or progression of cancer (cancer tissue) in a patient and or the progress of therapy in a patient or subject.

The term “histone-packaged supercoiled plasmid DNA” is used to describe a preferred component of protocells according to the present invention which utilize a preferred plasmid DNA which has been “supercoiled” (i.e., folded in on itself using a supersaturated salt solution or other ionic solution which causes the plasmid to fold in on itself and “supercoil” in order to become more dense for efficient packaging into the protocells). The plasmid may be virtually any plasmid which expresses any number of polypeptides or encode RNA, including small hairpin RNA/shRNA or small interfering RNA/siRNA, as otherwise described herein. Once supercoiled (using the concentrated salt or other anionic solution), the supercoiled plasmid DNA is then complexed with histone proteins to produce a histone-packaged “complexed” supercoiled plasmid DNA.

“Packaged” DNA herein refers to DNA that is loaded into protocells (either adsorbed into the pores or confined directly within the nanoporous silica core itself). To minimize the DNA spatially, it is often packaged, which can be accomplished in several different ways, from adjusting the charge of the surrounding medium to creation of small complexes of the DNA with, for example, lipids, proteins, or other nanoparticles (usually, although not exclusively cationic). Packaged DNA is often achieved via lipoplexes (i.e. complexing DNA with cationic lipid mixtures). In addition, DNA has also been packaged with cationic proteins (including proteins other than histones), as well as gold nanoparticles (e.g. NanoFlares—an engineered DNA and metal complex in which the core of the nanoparticle is gold).

Any number of histone proteins, as well as other means to package the DNA into a smaller volume such as normally cationic nanoparticles, lipids, or proteins, may be used to package the supercoiled plasmid DNA “histone-packaged supercoiled plasmid DNA”, but in therapeutic aspects which relate to treating human patients, the use of human histone proteins are preferably used. In certain aspects of the invention, a combination of human histone proteins H1, H2A, H2B, H3 and H4 in a preferred ratio of 1:2:2:2:2, although other histone proteins may be used in other, similar ratios, as is known in the art or may be readily practiced pursuant to the teachings of the present invention. The DNA may also be double stranded linear DNA, instead of plasmid DNA, which also may be optionally supercoiled and/or packaged with histones or other packaging components.

Other histone proteins which may be used in this aspect of the invention include, for example, H1F, H1F0, H1FNT, H1FOO, H1FX H1H1 HIST1H1A, HIST1H1B, HIST1H1C, HIST1H1D, HIST1H1E, HIST1H1T; H2AF, H2AFB1, H2AFB2, H2AFB3, H2AFJ, H2AFV, H2AFX, H2AFY, H2AFY2, H2AFZ, H2A1, HIST1H2AA, HIST1H2AB, HIST1H2AC, HIST1H2AD, HIST1H2AE, HIST1H2AG, HIST1H2AI, HIST1H2AJ, HIST1H2AK, HIST1H2AL, HIST1H2AM, H2A2, HIST2H2AA3, HIST2H2AC, H2BF, H2BFM, HSBFS, HSBFWT, H2B1, HIST1H2BA, HIST1HSBB, HIST1HSBC, HIST1HSBD, HIST1H2BE, HIST1H2BF, HIST1H2BG, HIST1H2BH, HIST1H2BI, HIST1H2BJ, HIST1H2BK, HIST1H2BL, HIST1H2BM, HIST1H2BN, HIST1H2BO, H2B2, HIST2H2BE, H3A1, HIST1H3A, HIST1H3B, HIST1H3C, HIST1H3D, HIST1H3E, HIST1H3F, HIST1H3G, HIST1H3H, HIST1H3I, HIST1H3J, H3A2, HIST2H3C, H3A3, HIST3H3, H41, HIST1H4A, HIST1H4B, HIST1H4C, HIST1H4D, HIST1H4E, HIST1H4F, HIST1H4G, HIST1H4H, HIST1H4I, HIST1H4J, HIST1H4K, HIST1H4L, H44 and HIST4H4.

The term “nuclear localization sequence” refers to a peptide sequence incorporated or otherwise crosslinked into histone proteins which comprise the histone-packaged supercoiled plasmid DNA. In certain embodiments, protocells according to the present invention may further comprise a plasmid (often a histone-packaged supercoiled plasmid DNA) which is modified (crosslinked) with a nuclear localization sequence (note that the histone proteins may be crosslinked with the nuclear localization sequence or the plasmid itself can be modified to express a nuclear localization sequence) which enhances the ability of the histone-packaged plasmid to penetrate the nucleus of a cell and deposit its contents there (to facilitate expression and ultimately cell death (apoptosis). These peptide sequences assist in carrying the histone-packaged plasmid DNA and the associated histones into the nucleus of a targeted cell whereupon the plasmid will express peptides and/or nucleotides as desired to deliver immune, therapeutic and/or diagnostic molecules (polypeptide and/or nucleotide) into the nucleus of the targeted cell. Any number of crosslinking agents, well known in the art, may be used to covalently link an antigenic peptide to the lipid bilayer or other components of the MANPS, or a nuclear localization sequence to a histone protein (often at a lysine group or other group which has a nucleophilic or electrophilic group in the side chain of the amino acid exposed pendant to the polypeptide) which can be used to introduce the histone packaged plasmid into the nucleus of a cell. Alternatively, a nucleotide sequence which expresses the nuclear localization sequence can be positioned in a plasmid in proximity to that which expresses histone protein such that the expression of the histone protein conjugated to the nuclear localization sequence will occur thus facilitating transfer of a plasmid into the nucleus of a targeted cell.

Proteins gain entry into the nucleus through the nuclear envelope. The nuclear envelope consists of concentric membranes, the outer and the inner membrane. These are the gateways to the nucleus. The envelope consists of pores or large nuclear complexes. A protein translated with a NLS will bind strongly to importin (aka karyopherin), and together, the complex will move through the nuclear pore. Any number of nuclear localization sequences may be used to introduce histone-packaged plasmid DNA into the nucleus of a cell. Preferred nuclear localization sequences include H₂N-GNQSSNFGPMKGGNFGGRSSGPYGGGGQYFAKPRNQGGYGGC-COOH (SEQ LD NO: 1), RRMKWKK (SEQ ID NO: 2), PKKKRKV (SEQ ID NO: 3), and KR[PAATKKAGQA]KKKK (SEQ ID NO:4), the NLS of nucleoplasmin, a prototypical bipartite signal comprising two clusters of basic amino acids, separated by a spacer of about 10 amino acids. Numerous other nuclear localization sequences are well known in the art. See, for example, LaCasse, et al., Nuclear localization signals overlap DNA- or RNA-binding domains in nucleic acid-binding proteins. Nucl. Acids Res., 23, 1647-1656 1995); Weis, K. Importins and exportins: how to get in and out of the nucleus [published erratum appears in Trends Biochem Sci 1998 July; 23(7):235]. TIBS, 23, 185-9 (1998); and Murat Cokol, Raj Nair & Burkhard Rost, “Finding nuclear localization signals”, at the website ubic.bioc.columbia.edu/papers/2000 nls/paper.html#tab2.

The terms “co-administer” and “co-administration” are used synonymously to describe the administration of at least one of the MANPS compositions according to the present invention in combination with at least one other agent, often at least one additional antibiotic or antiviral agent (as otherwise described herein), which are specifically disclosed herein in amounts or at concentrations which would be considered to be effective amounts at or about the same time. While it is preferred that co-administered compositions/agents be administered at the same time, agents may be administered at times such that effective concentrations of both (or more) compositions/agents appear in the patient at the same time for at least a brief period of time. Alternatively, in certain aspects of the present invention, it may be possible to have each co-administered composition/agent exhibit its inhibitory effect at different times in the patient, with the ultimate result being the inhibition and treatment of cancer, especially including hepatocellular or liver cancer, among others, as well as the reduction or inhibition of other disease states, conditions or complications. Of course, when more than disease state, infection or other condition is present, the present compounds may be combined with other agents to treat that other infection or disease or condition as required.

The terms “targeting ligand” and “targeting active species” are used to describe a compound or moiety (preferably an antigen) which is complexed or preferably covalently bonded to the surface of a MANPS according to the present invention which binds to a moiety on the surface of a cell to be targeted so that the MANPS may selectively bind to the surface of the targeted cell and deposit its contents into the cell. The targeting active species for use in the present invention is preferably a targeting peptide as otherwise described herein, a polypeptide including an antibody or antibody fragment, an aptamer, or a carbohydrate, among other species which bind to a targeted cell. Targeting ligands are exemplified in Example 3.

A “targeting peptide” is one type of targeting ligand and is a peptide which binds to a receptor or other polypeptide in a target cell (e.g. a cancer cell) and allows the targeting of MANPS according to the present invention to particular cells which express a peptide (be it a receptor or other functional polypeptide) to which the targeting peptide binds. Targeting peptides may be complexed or preferably, covalently linked to the lipid bilayer through use of a crosslinking agent as otherwise described herein.

The terms “fusogenic peptide” and “endosomolytic peptide” are used to describe a peptide which is optionally and preferred crosslinked onto the lipid bilayer surface of the protocells according to the present invention. Fusogenic peptides are incorporated onto protocells in order to facilitate or assist escape from endosomal bodies and to facilitate the introduction of protocells into targeted cells to effect an intended result (therapeutic and/or diagnostic as otherwise described herein). Representative and preferred fusogenic peptides for use in protocells according to the present invention include H5WYG peptide, H₂N-GLFHAIAHFIHGGWHGLIHGWYGGC-COOH (SEQ ID. NO: 5) or an 8 mer polyarginine (H₂N-RRRRRRRR-COOH, SEQ ID NO:), among others known in the art.

The term “cross-linking agent” is used to describe a compound which may be used to covalently link various components according to the present invention to each other, such as a bifunctional compound of varying length containing two different functional groups. Crosslinking agents according to the present invention may contain two electrophilic groups (to react with nucleophilic groups on peptides of oligonucleotides, one electrophilic group and one nucleophilic group or two two nucleophlic groups). The crosslinking agents may vary in length depending upon the components to be linked and the relative flexibility required. Crosslinking agents are used to anchor targeting and/or fusogenic peptides to the phospholipid bilayer, to link nuclear localization sequences to histone proteins for packaging supercoiled plasmid DNA and in certain instances, to crosslink lipids in the lipid bilayer of the protocells. There are a large number of crosslinking agents which may be used in the present invention, many commercially available or available in the literature. Preferred crosslinking agents for use in the present invention include, for example, 1-Ethyl-3-[3-dimethylaminopropyl]carbodiimide hydrochloride (EDC), succinimidyl 4-[N-maleimidomethyl]cyclohexane-1-carboxylate (SMCC), N-[β-Maleimidopropionic acid] hydrazide (BMPH), NHS-(PEG)ₙ-maleimide, succinimidyl-[(N-maleimidopropionamido)-tetracosaethyleneglycol]ester (SM(PEG)₂₄), and succinimidyl 6-[3′-(2-pyridyldithio)-propionamido]hexanoate (LC-SPDP), among others.

In one embodiment, in order to induce both humoral and cellular immune responses and to tune the magnitude of these responses, MANPs are loaded with cocktails of antigens and, if necessary, immunostimulatory molecule(s) and encapsulated within a SLB, which can be further modified with ligands that promote uptake by APCs and/or cytosolic release of encapsulated antigen(s).

As explained above, another aspect of the invention relates to the use of aerosol-assisted evaporation-induced self-assembly to provide mesoporous oxide nanoparticles that can be stably loaded with high concentrations of various antigens and engineered for burst or sustained release profiles. Aerosol-assisted evaporation-induced self-assembly enables modification of a nanoparticle surface with various targeting ligands and promotes effective uptake by antigen-presenting cells. Antigen-loaded mesoporous oxide nanoparticles induce antigen-specific humoral and cellular immune responses.

In a preferred embodiment, the present invention is directed to mesoporous alum nanoparticles (MANPS) to which antigen has been adsorbed.

In still another embodiment, the invention is directed to MANPS in which antigen has been cross-linked to facilitate antigen orientation and dense, repetitive presentation to facilitate an immune response.

In still another embodiment, the invention is directed to MANPS which are loaded with antigen (as cargo) and encapsulated within a supported lipid bilayer (SLP) and which are preferably modified with ligands that facilitate uptake of the nanoparticles by antigen-presenting cells (APCs) and/or cytosolic dispersion of antigen.

In still another embodiment, MANPS according to the present invention may be used to simultaneously deliver cocktails of physicochemically disparate antigens and, if necessary, immunostimulatory molecules.

Accordingly, as described above, in certain aspects, the present invention is directed to a cell-targeting mesoporous alum nanoparticle comprising a nanoporous alum with an optional supported lipid bilayer; at least one antigen; and at least one further component selected from the group consisting of
a cell targeting species;
a ligand that facilitates uptake of the nanoparticles by antigen-presenting cells (APCs) and/or cytosolic dispersion of antigen;
a fusogenic peptide that promotes endosomal escape of nanoparticles and encapsulated DNA, and other cargo comprising at least one additional cargo component (other than the antigen) selected from the group consisting of double stranded linear DNA or a plasmid DNA;
at least one drug;
an imaging agent,
small interfering RNA, small hairpin RNA, microRNA, or a mixture thereof,
wherein one of said cargo components is optionally conjugated further with a nuclear localization sequence.

In certain embodiments, nanoparticles according to embodiments of the invention comprise a nanoporous alum-based core with a supported lipid bilayer; a cargo comprising at least one antigen which facilitates an immune response in a subject or patient; and optionally at least one agent selected from an optional therapeutic agent such as a traditional small molecule, a macromolecular cargo (e.g. siRNA such as 5565, 57824 and/or s10234, among others, shRNA and/or a packaged plasmid DNA (in certain embodiments-histone packaged).

The aforementioned macromolecular cargo is disposed within the nanoporous alum core (preferably supercoiled as otherwise described herein) in order to more efficiently package the DNA into protocells as a cargo element) and is optionally modified with a nuclear localization sequence to assist in localizing/presenting the plasmid within the nucleus of a targeted cell. This enables expressed proteins to function therapeutically or as a reporter (e.g. fluorescent green protein, fluorescent red protein, among others, as otherwise described herein) in diagnostic applications.

Nanoparticles according to the present invention optionally include a targeting peptide which targets cells for introduction of the antigen such that binding of the nanoparticle to the targeted cells is specific and enhanced and a fusogenic peptide that promotes endosomal escape of nanoparticles and encapsulated DNA. Nanopaticles according to the present invention may be used to generate an immune response, in therapy or diagnostics, more specifically to reduce the likelihood of pathogens (bioterrorism), cancer and other diseases, including microbial infections, including bacterial and viral infections. In other aspects of the invention, nanoparticles use novel binding peptides which selectively bind to tissue to target an immune response, for therapy and/or diagnosis of an infection and/or disease state.

In another preferred embodiment, the invention provides a mesoporous alum nanoparticle which has a pore size of approximately 1 nm to approximately 75 nm (thus, mesoporous nanoparticles as used herein distinguish over the IUPAC definition of mesopores, unless otherwise indicated) and which is loaded with one or more antigens selected from the group consisting of a glycoprotein or lipoprotein derived from a Category A or B biothreat bacteria, virus or toxin. Such bacteria, viruses or toxins include, but are not limited to, E. coli O157:H7 lipopolysaccharide (LPS), anthrax protective antigen (PA), soluble Nipah virus glycoprotein (sG), ricin toxin A-chain (RTA), ovalbumin (OVA), F. tularensis lipopolysaccharide, recombinant Bacillus anthracis protective antigen, recombinant botulinum neurotoxin type A (BoNT-A) light chain (LC), Zaire Ebola virus glycoprotein (sGP), filo- and arenavirus antigens, Ig1C, PA, sGP, sGP1, RTA, and BoNT-A LC, formalin-inactivated Venezuelan equine encephalitis virus vaccine strain TC-83 and lysozyme (LSZ).

Preferably, the mesoporous alum nanoparticle described in the preceding paragraph is encapsulated within a supported lipid bi-layer (e.g. a lipid bi-layer comprised of 1,2-dioleoyl-sn-glycerol-3-phosphocholine (DOPC)), the nanoparticle further comprises an immunostimulatory RNA (isRNA), the antigen comprises about 50% to about 70% by weight of the nanoparticle and the nanoparticle is made by aerosol-assisted evaporation-induced self-assembly.

Notably, in certain embodiments of the mesoporous alum nanoparticles of the invention, the antigen comprises about 50% to about 70% by weight of the nanoparticle and the nanoparticle is made by aerosol-assisted evaporation-induced self-assembly.

Other aspects of embodiments of the present invention are directed to pharmaceutical compositions. Pharmaceutical compositions according to the present invention comprise a population of nanoparticles as otherwise described herein which may be the same or different and are formulated in combination with a pharmaceutically acceptable carrier, additive or excipient. The nanoparticles may be formulated alone or in combination with a bioactive agent (such as an antibiotic, an additional bioactive agent or an antiviral agent) depending upon the disease to be prevented and the route of administration (as otherwise described herein). These compositions comprise nanoparitcles as modified for a particular purpose (e.g. generating an immune response, etc. Pharmaceutical compositions comprise an effective population of nanoparticles for a particular purpose and route of administration in combination with a pharmaceutically acceptable carrier, additive or excipient.

An embodiment of the present invention also relates to methods of utilizing the novel nanoparticles as described herein to generate an immune response. Thus, in alternative embodiments, the present invention relates to a method of eliciting an immune response in a host or patient (preferably, both a humoral and cell mediated response), preventing and/or reducing the likelihood of disease in a subject or patient at risk for said disease, optionally treating a disease and/or condition comprising administering to a patient or subject in need an effective amount of a pharmaceutical composition as otherwise described herein. The pharmaceutical compositions according to the present invention are particularly useful for eliciting an immune response and/or preventing and/or reducing the likelihood of a number of disease states and/or conditions, especially diseases which are caused by microbes, such as bacteria and viruses, especially pathogenic/virulent bacteria and viruses.

As discussed in detail above, the porous nanoparticle core of the present invention can include porous nanoparticles having at least one dimension, for example, a width or a diameter of about 3,000 nm or less, about 1,000 nm or less, about 500 nm or less, about 200 nm or less. Preferably, the nanoparticle core is spherical with a preferred diameter of about 500 nm or less, more preferably about 8-10 nm to about 200 nm. In embodiments, the porous particle core can have various cross-sectional shapes including a circular, rectangular, square, or any other shape. In certain embodiments, the porous particle core can have pores with a mean pore size ranging from about lnm to about 75 nm, often about 2 nm to about 30 nm, although the mean pore size and other properties (e.g., porosity of the porous particle core) are not limited in accordance with various embodiments of the present teachings.

In general, MANPS according to the present invention are biocompatible. Antigens, drugs and other cargo components are often loaded by adsorption and/or capillary filling of the pores of the particle core up to approximately 50% by weight of the final protocell (containing all components). In certain embodiments according to the present invention, the loaded cargo can be released from the porous surface of the particle core (mesopores), wherein the release profile can be determined or adjusted by, for example, the pore size, the surface chemistry of the porous particle core, the pH value of the system, and/or the interaction of the porous particle core with the surrounding lipid bilayer(s) as generally described herein.

In the present invention, the porous nanoparticle core used to prepare the protocells can be tuned in to be hydrophilic or progressively more hydrophobic as otherwise described herein and can be further treated to provide a more hydrophilic surface. For example, mesoporous silica particles can be further treated with ammonium hydroxide and hydrogen peroxide to provide a higher hydrophilicity. In preferred aspects of the invention, the lipid bilayer is fused onto the porous particle core to form the protocell. Protocells according to the present invention can include various lipids in various weight ratios, preferably including 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof.

The lipid bilayer which is used to prepare protocells according to the present invention can be prepared, for example, by extrusion of hydrated lipid films through a filter with pore size of, for example, about 100 nm, using standard protocols known in the art or as otherwise described herein. The filtered lipid bilayer films can then be fused with the porous particle cores, for example, by pipette mixing. In certain embodiments, excess amount of lipid bilayer or lipid bilayer films can be used to form the protocell in order to improve the protocell colloidal stability.

In certain diagnostic embodiments, various dyes or fluorescent (reporter) molecules can be included in the protocell cargo (e.g., as expressed by plasmid DNA) or attached to the porous particle core and/or the lipid bilayer for diagnostic purposes. For example, the porous particle core can be a silica core or the lipid bilayer and can be covalently labeled with FITC (green fluorescence), while the lipid bilayer or the particle core can be covalently labeled with FITC Texas red (red fluorescence). The porous particle core, the lipid bilayer and the formed protocell can then be observed by, for example, confocal fluorescence for use in diagnostic applications. In addition, as discussed herein, plasmid DNA can be used as cargo in protocells according to the present invention such that the plasmid may express one or more fluorescent proteins such as fluorescent green protein or fluorescent red protein which may be used in diagnostic applications.

In various embodiments, the MANPS protocell is used in a synergistic system where the lipid bilayer fusion or liposome fusion (i.e., on the porous particle core) is loaded and sealed with various cargo components with the pores (mesopores) of the particle core, thus creating a loaded protocell useful for cargo delivery across the cell membrane of the lipid bilayer or through dissolution of the porous nanoparticle, if applicable. In certain embodiments, in addition to fusing a single lipid (e.g., phospholipids) bilayer, multiple bilayers with opposite charges can be successively fused onto the porous particle core to further influence cargo loading and/or sealing as well as the release characteristics of the final MANPS protocell.

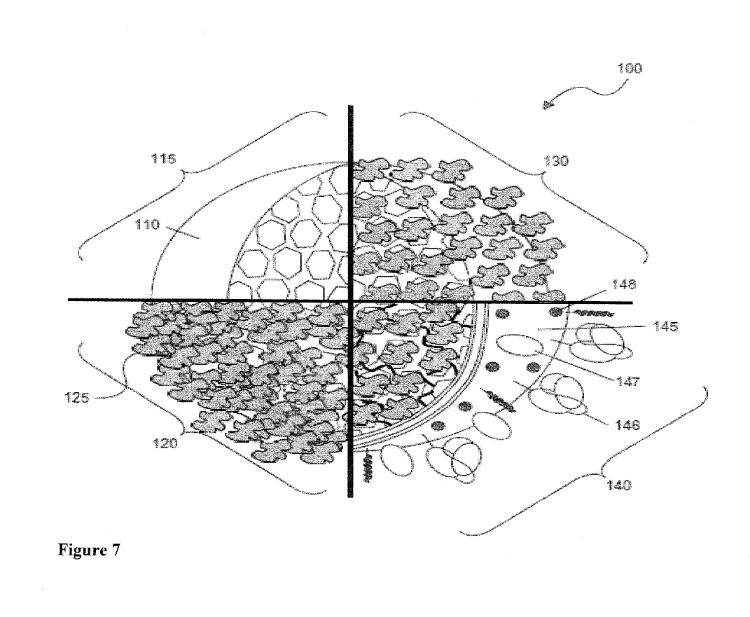

FIG. 7 shows a schematic perspective side view of an embodiment of a protocell. In FIG. 7, the protocell is divided into quadrants, each quadrant illustrating different embodiments of a protocell. In this embodiment, protocell 100 includes particle core 110 illustrated in quadrant 115. Core 110, in one embodiment, is a core of porous nanoparticles. Representative materials for nanoparticles include inorganic materials such as silica, alumina, titania and zirconia material as well as organic material (e.g., polymeric material) or a combination of inorganic and organic material. In the embodiment shown, core 110 is nanoparticles of aluminum hydroxide or aluminum sulfate. In another embodiment, core 110 is nanoparticles of silica. Core 110 includes particles collectively defining a body having a dimension or diameter on the order of 500 nanometers (nm) or less (e.g., 30 nm to 100 nm or 5 nm to 200 nm or 500 nm or 20 nm to 200 nm). One example is a diameter range of 20 nm to 200 nm with 150 nm being a mean or median diameter. FIG. 7 illustrates core 110 having a circular shape. It is appreciated that core can have other shapes including, but not limited to, oval, rectangular and irregular (i.e., random and not generally classifiable shape). For example, particles with mean diameters between 20-150 nm would preferentially stay in systemic circulation and are therefore more ideal when used in conjunction with a targeting ligand (providing longer circulation time enhances chance of contact with target). Particles with mean diameters 200-500 nm are rapidly cleared by the liver and reticuloendothelial system such that they may be ideal for delivery to lympthatics and spleen where the majority of immune cells reside. Alternatively, larger particles would tend to arrest in immediate tissues and could serve as a long-lasting depot for antigen/adjuvant (essentially replacing the need for multiple or booster shots but simply serving as a local slow-release vaccine formulation).

In one embodiment, protocells, such as protocell 100, are characterized by containing mesopores in core 110. These pores (at least one, but often a large number) may be found intersecting a surface of a nanoparticle core (by having one or both ends of the pore appearing on the surface of the nanoparticle) or the internal to a nanostructure with at least one or more mesopore interconnecting with surface mesoporous of the nanoparticle. Interconnecting pores of smaller size are found internal to the surface of mesopore. An overall range of pore size of mesopores can be about 0.03 nanometers to 50 nanometers or more in diameter. In one embodiment, pore sizes of mesopores range from about 2 nanometers to 30 nanometers. Core 110 representatively has pores with a mean or median pore size ranging from about 1 nm to 30 nm. Pores may be monosized or bimodal or graded, and ordered or disordered. FIG. 7 appears to indicate that an outer surface of core 110 is solid or impermeable. It is appreciated that a porous nature of the core may extend to an outer surface.

Mesopores (IUPAC definition 2 nm to 50 nm in diameter) are molded or formed by templating agents including surfactants, block copolymers, molecules, macromolecules, emulsions, latex beads or nanoparticles. In one embodiment, core particles of generally spherical shape are formed by generating an aerosol dispersion of the templating agent and the core material (e.g., aluminum chloride (AlCl₃.6H₂O) for aluminum hydroxide nanoparticle; potassium alum (KARSO₄)₂.12H₂O) for aluminum sulfate nanoparticle; and tetraethyl orthosilicate (TEOS) for a silica nanoparticle) in a tubular reactor and then drying the particles. See, e.g., “Aerosol-assisted self-assembly of mesostructured spherical nanoparticles,” Lu, Y., et al., Nature, Vol. 398, 223-26 (1999), incorporated herein by reference. Generally, in an aerosol-assisted evaporation-induced self-assembly (EISA) process, a dilute solution of a metal salt or metal alkoxide is dissolved in an alcohol/water solvent along with ionic or non-ionic surfactants, block copolymers (e.g., Pluronic P-123, a triblock copolymer manufactured by BASF Corporation). The resulting solution is then aerosolized with a carrier gas and introduced into a laminar flow reactor.

Surfactants/templates can be extracted using either acidified ethanol or thermal calcination to yield mesoporous hydroxides (boehmite AlO(OH) or gibbsite Al(OH)₃) or sulfates (alum). The process may be used to form particles with systemically variable pore sizes (e.g., 2 nm to 50 nm), pore geometrics (e.g., hexagonal, cubic, lamellar, cellular) and surface areas (100 m²/g to greater than 1200 m²/g). A representative mesoporous alum nanoparticle (MANP) with a surface area on the order of about 500 m²/g and 10 nm pores can be templated by using a block copolymer of Pluronic P-123. In addition, processes could lead to micropores (IUPAC definition less than 2 nanometers in diameter) if a templating moiety in an aerosol process is not used. Processes can also move to macropores, i.e., pores greater than 50 nm in diameter.

Pore surface chemistry of a nanoparticle material can be diverse. Attractive electrostatic interactions or hydrophobic interactions tend to control or enhance a loading capacity and a release rate. Higher surface areas can lead to higher loading of a cargo through these attractive interactions. In one embodiment, a porous nanoparticle core can be tuned in to be hydrophilic or progressively more hydrophobic and can be further treated to provide a more hydrophilic surface. For example, mesoporous silica particles can be further treated with ammonium hydroxide and hydrogen peroxide to provide a higher hydrophilicity.

Mesoporous alum nanoparticles (MANPs) are naturally negatively-charged (ζ=−20 mV in 0.5×PBS, pH 7.4). In one embodiment, alum nanoparticles can be soaked in a 10 mol % solution of the amine-containing silane, (3-aminopropyl)triethoxysilane (APTES) for six hours at room temperature. The pore network of resulting particles will contain primary amine groups and should, therefore, readily adsorb the majority of antigens. To facilitate absorption of amphiphilic antigens (e.g., F. tularensis lipopolysaccharide), MANPs can be modified with hexamethyldisilazane (HMDS) by soaking the particles in a 6 mol % solution for six hours at room temperature. To facilitate antigen cross-linking, MANPs can be soaked in a 5 mol % solution of (3-mercaptopropyl)trimethoxysilane (MPTS) for two hours at room temperature. Fourier transform infrared (FTIR) spectroscopy can be used to determine the overall zeta potential of APTES, HMDS, and MPTS-modified MANPs and quantify the approximate density of primary amine (—NH3), methyl (—CH3), and sulfhydryl (—SH) moieties.

Core particles dissolution rate may be varied or tuned by the degree of condensation. A fully condensed inorganic core structure (e.g., alum or silica) will dissolve in vivo at a slower rate than a less condensed structure.

Disposed within and/or adsorped to core 110 of, for example, a mesoporous alum nanoparticle (MANP) of protocell 100 in FIG. 7, in one embodiment, is cargo 125 including an adjuvant and/or one or more antigens. MANPs can be loaded with a variety of antigens including but not limited to glycoproteins and lipoproteins, derived from Category A or B bacteria, viruses, and toxins; lipopolysaccharide (LPS) isolated from the live vaccine strain (LVS) of F. tularensis (Ft), subsp. holarctica from BEI Resources; recombinant Bacillus anthracis protective antigen (PA) from EMD Millipore; recombinant botulinum neurotoxin type A (BoNT-A) light chain (LC) from R&D Systems; and deglycosylated ricin toxin A-chain (RTA) from Sigma-Aldrich. Recombinant Ft intracellular growth locus C (Ig1C) with a C-terminal (His)₆ affinity tag synthesized by Proteos, Inc.; soluble Zaire Ebola virus glycoprotein (sGP) and soluble GP1 (sGP1) derived from the Lassa fever virus GPC gene using a technique similar to the procedure used by Negrete, et al. for the production of soluble Nipah virus glycoprotein.

A fusion and synergistic loading mechanism can be included for cargo delivery. For example, cargo can be loaded, encapsulated, or sealed, synergistically through liposome fusion on the porous particles. The cargo can include, for example, small molecule drugs (e.g. especially including anticancer drugs and/or antiviral drugs such as anti-HBV or anti-HCV drugs), peptides, proteins, antibodies, DNA (especially plasmid DNA, including the preferred histone-packaged super coiled plasmid DNA), RNAs (including shRNA and siRNA (which may also be expressed by the plasmid DNA incorporated as cargo within the protocells) fluorescent dyes, including fluorescent dye peptides which may be expressed by the plasmid DNA incorporated within the protocell.

In embodiments according to the present invention, the cargo can be loaded into the pores (mesopores) of the porous particle cores to form the loaded MANPS protocell. In various embodiments, any conventional technology that is developed for liposome-based drug delivery, for example, targeted delivery using PEGylation, can be transferred and applied to the protocells of the present invention.

As discussed above, electrostatics and pore size can play a role in cargo loading. For example, porous nanoparticles can carry a negative charge and the pore size can be tunable from about 2 nm to about 10 nm or more. Negatively charged nanoparticles can have a natural tendency to adsorb positively charged molecules and positively charged nanoparticles can have a natural tendency to adsorb negatively charged molecules. In various embodiments, other properties such as surface wettability (e.g., hydrophobicity) can also affect loading cargo with different hydrophobicity.

In various embodiments, the cargo loading can be a synergistic lipid-assisted loading by tuning the lipid composition. For example, if the cargo component is a negatively charged molecule, the cargo loading into a negatively charged silica can be achieved by the lipid-assisted loading. In certain embodiments, for example, a negatively species can be loaded as cargo into the pores of a negatively charged silica particle when the lipid bilayer is fused onto the silica surface showing a fusion and synergistic loading mechanism. In this manner, fusion of a non-negatively charged (i.e., positively charged or neutral) lipid bilayer or liposome on a negatively charged mesoporous particle can serve to load the particle core with negatively charged cargo components. The negatively charged cargo components can be concentrated in the loaded protocell having a concentration exceed about 100 times as compared with the charged cargo components in a solution. In other embodiments, by varying the charge of the mesoporous particle and the lipid bilayer, positively charged cargo components can be readily loaded into protocells.

Once produced, the loaded MANPS can have a cellular uptake for cargo delivery into a desirable site after administration. For example, the cargo-loaded protocells can be administered to a patient or subject and the protocell comprising a targeting peptide can bind to a target cell and be internalized or uptaken by the target cell, for example, in a subject or patient. Due to the internalization of the cargo-loaded MANPS protocells in the target cell, cargo components can then be delivered into the target cells. In certain embodiments the cargo is an antigenic peptide or other small molecule, which can be delivered directly into the target cell for therapy. In other embodiments, negatively charged DNA or RNA (including shRNA or siRNA), especially including a DNA plasmid which is preferably formulated as histone-packaged supercoiled plasmid DNA preferably modified with a nuclear localization sequence can be directly delivered or internalized by the targeted cells. Thus, the DNA or RNA can be loaded first into a MANPS and then into then through the target cells through the internalization of the loaded protocells.

As discussed, the cargo loaded into and delivered by the protocell to targeted cells includes antigens, small molecules or drugs (especially antimicrobial agents or antiviral agents), bioactive macromolecules (bioactive polypeptides or RNA molecules such as shRNA and/or siRNA as otherwise described herein) or histone-packaged supercoiled plasmid DNA which can express a therapeutic or diagnostic peptide or a therapeutic RNA molecule such as shRNA or siRNA, wherein the histone-packaged supercoiled plasmid DNA is optionally and preferably modified with a nuclear localization sequence which can localize and concentrate the delivered plasmid DNA into the nucleus of the target cell. As such, loaded MANPS can deliver their cargo into targeted cells for eliciting an immune response, for therapy or diagnostics.

In various embodiments according to the present invention, the MANPS and/or the loaded protocells can provide a targeted delivery methodology for selectively delivering the MANPS or the cargo components to targeted cells. For example, a surface of the lipid bilayer can be modified by a targeting active species that corresponds to the targeted cell. The targeting active species may be a targeting peptide as otherwise described herein, a polypeptide including an antibody or antibody fragment, an aptamer, a carbohydrate or other moiety which binds to a targeted cell.

For example, by providing a targeting active species (preferably, a targeting peptide) on the surface of the loaded protocell, the protocell selectively binds to the targeted cell in accordance with the present teachings. In one embodiment, by conjugating an exemplary targeting peptide or analog otherwise described herein that targets cells, a large number of the cargo-loaded protocells can be recognized and internalized by this specific cancer cells due to the specific targeting of the binding peptide with the target cells. In most instances, if the protocells are conjugated with the targeting peptide, the MANPS will selectively bind to the cells and no appreciable binding to the non-targeted cells occurs.

Once bound and taken up by the target cells, the loaded protocells can release cargo components from the porous particle and transport the released cargo components into the target cell. For example, sealed within the protocell by the liposome fused bilayer on the porous particle core, the cargo components can be released from the pores of the lipid bilayer, transported across the protocell membrane of the lipid bilayer and delivered within the targeted cell. In embodiments according to the present invention, the release profile of cargo components in protocells can be more controllable as compared with when only using liposomes as known in the prior art. The cargo release can be determined by, for example, interactions between the porous core and the lipid bilayer and/or other parameters such as pH value of the system. For example, the release of cargo can be achieved through the lipid bilayer, through dissolution of the porous silica; while the release of the cargo from the protocells can be pH-dependent.

In certain embodiments, the pH value for cargo is often less than 7, preferably about 4.5 to about 6.0, but can be about pH 14 or less. Lower pHs tend to facilitate the release of the cargo components significantly more than compared with high pHs. Lower pHs tend to be advantageous because the endosomal compartments inside most cells are at low pHs (about 5.5), but the rate of delivery of cargo at the cell can be influenced by the pH of the cargo. Depending upon the cargo and the pH at which the cargo is released from the protocell, the release of cargo can be relative short (a few hours to a day or so) or a span for several days to about 20-30 days or longer. Thus, the present invention may accommodate immediate release and/or sustained release applications from the MANPS themselves.

In certain embodiments, the inclusion of surfactants can be provided to rapidly rupture the lipid bilayer, transporting the cargo components across the lipid bilayer of the protocell as well as the targeted cell. In certain embodiments, the phospholipid bilayer of the protocells can be ruptured by the application/release of a surfactant such as sodium dodecyl sulfate (SDS), among others to facilitate a rapid release of cargo from the protocell into the targeted cell. In certain embodiments, the rupture of the lipid bilayer can in turn induce immediate and complete release of the cargo components from the pores of the particle core of the protocells. In this manner, the protocell platform can provide versatile delivery systems as compared with other delivery systems in the art. For example, when compared to delivery systems using nanoparticles only, the disclosed protocell platform can provide a simple system and can take advantage of the low toxicity and immuneity of liposomes or lipid bilayers along with their ability to be PEGylated or to be conjugated to extend circulation time and effect targeting. In another example, when compared to delivery systems using liposome only, the protocell platform can provide a more stable system and can take advantage of the mesoporous core to control the loading and/or release profile.

In addition, the lipid bilayer and its fusion on porous particle core can be fine-tuned to control the loading, release, and targeting profiles and can further comprise fusogenic peptides and related peptides to facilitate delivery of the protocells for greater therapeutic and/or diagnostic effect. Further, the lipid bilayer of the MANPS protocells can provide a fluidic interface for ligand display and multivalent targeting, which allows specific targeting with relatively low surface ligand density due to the capability of ligand reorganization on the fluidic lipid interface. Furthermore, the disclosed protocells can readily enter targeted cells while empty liposomes without the support of porous particles cannot be internalized by the cells.

Referring to FIG. 7, mesoporous silica nanoparticle (MSNP) core 110 of protocell 100 may representatively be loaded up to approximately 55% by weight of the final protocell (containing all components) depending on the size of the cargo. A cargo such as an adjuvant and/or a protein antigen may be loaded (e.g., adsorbed) into/on protocell 100 by capillary filling of the pores of core 110. The immersion tends to trap the cargo in pores of such the particles that make up core 110. In one embodiment, aloading capacity is a function of cargo size and charge, as well as nanoparticle charge, pore size, and available internal surface area/pore volume. These nanoparticle parameters can be independently altered in order to optimize loading capacity of a mesoporous nanoparticle).

Alternatively, rather than immersing a particle core in a solution of the cargo, in another embodiment, the particle core may be assembled around the cargo. One way this may be accomplished is by combining precursors to the particle core with an adjuvant and/or protein antigen(s) and spray drying the combination. Representatively, for a particle core of mesoporous silica particle, the precursors may include hydrochloric acid (HCl), a surfactant such as catrimonium bromide (CTAB) and tetraethylorthosilade (TEOS) that may be combined with a adjuvant and/or protein antigen(s).

Referring to the mesoporous alum nanoparticle (MANP) illustrated in FIG. 7, the nanoparticle may be cargo loaded in three different general formulations illustrated in quadrants of the nanoparticle of FIG. 7. Referring to protocell 100, quadrant 115 illustrates a quadrant of only the porous nanoparticle (i.e., core 110 free of cargo). Quadrant 120 illustrates a formulation where antigens 125 are randomly adsorbed to core 110.

In one embodiment, (3-aminopropyl)triethoxysilane (APTES)-modified MANPs are utilized for random adsorption of Ig1C, PA, sGP, sGP1, RTA, and BoNT-A LC and HMDS-modified MANPs for random adsorption of LPS; Ig1C and LPS may be co-loaded using MANPs modified with both APTES and HMDS. To promote antigen adsorption, MANPs can be soaked in an aqueous solution of the desired antigen(s) for 12 hours at 4° C. and washed three times with 1×PBS to remove unencapsulated antigen. MANPs with a high degree of framework condensation will be used for random adsorption of antigen(s) since resulting particles will likely act as an antigen depot and should, therefore, release antigen over a period of one to two weeks to maximize interaction times between antigen and APCs. Other aminosilanes such as (3-aminopropyl)-diethoxy-methylsilane (APDEMS), (3-aminopropyl)-dimethyl-ethoxysilane (APDMES) and (3-aminopropyl)-trimethoxysilane (APTMS) can be substituted for APTES, or a combination of aminosilanes can be used.

Quadrant 130 of protocell 100 illustrates MANPs to which antigens are cross-linked to facilitate antigen orientation and high-density presentation. To cross-link Ig1C, PA, sGP, sGP1, RTA, and BoNT-A LC to MANPs, MPTS-modified particles can be incubated with a 10-fold molar excess of the non-cleavable amine-to-sulfhydryl cross-linker, sulfosuccinimidyl-4-(N-maleimidomethyl)cyclohexane-1-carboxylate (sulfo-SMCC) or the reducible amine-to-sulfhydryl cross-linker, sulfosuccinimidyl 6-(3′-[2-pyridyldithio]-propionamido)hexanoate (sulfo-LC-SPDP) for one hour at room temperature; cross-linker-activated particles will then be incubated with 1 mg/mL of antigen overnight at 4° C. To cross-link LP S, the sulfhydryl-to-hydroxyl cross-linker, N-(p-maleimidiophenyl)isocyanate (PMPI) can be employed. The pore network can also be modified with Ni(II) complexes to orient and immobilize proteins with (His)₆ affinity tags. Although it is anticipated that a high density of surface-exposed antigen will trigger maximal uptake by APCs, if necessary, the reaction stoichiometry can be varied to control the density of cross-linked antigens. MANPs with a high degree of framework condensation will be used in formulations with cross-linked antigen(s).

Quadrant 140 of protocell 100 illustrates MANPs encapsulated (surrounded/enveloped) in one or more lipid bilayers to give the protocell a core-cladding structure. Representatively, any lipid polymer that is used in liposomes may also be used as a material from lipid bilayer 120. Representative lipids for use include, for example, 1,2-dioleoyl-5″-glycero-3-phosphocholine (DOPC), 1,2-dipalmitoyl-5″-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), glycero-3-phospho-(r-rac-glycerol) (DOPG), 1,2-dioleoyks77-glycero-3-phosphoethanolamine (DOPE), 1phosphoethanolamine (DPPE), 1,2-dioleoyl-src-glycero-3-phosphoethanolamine-N-[methoxy(polyethylene glycol)-2000] (18:1 PEG-2000PE), 1,2-dipalmitoyl-OT-glycero-3-phosphoethanolamine-N-[methoxy(polyethylene glycol)-2000] (16:0 PEG-2000 PE), 1-01eoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-5>7-glycero-3-phosphocholine (16:0-12:0 NBD PC), cholesterol and mixtures/combinations thereof. Cholesterol 160, not technically a lipid, but presented as a lipid for purposes of an embodiment may be incorporated in lipid bilayer(s) 120 in order to enhance structural integrity of the bilayer. These lipids are all readily available commercially from Avanti Polar Lipids, Inc. (Alabaster, Ala., USA). DOPE and DPPE are particularly useful for conjugating (through an appropriate cross-linker) peptides, polypeptides, including antibodies, RNA and DNA through the amine group on the lipid. Representative lipid bilayer 120 includes a mixture of lipids such as a weight ratio of 5 percent DOPE, 5 percent PEG 130, 30 percent cholesterol 160 and 60 percent DOPC or DPPC (by weight).

A charge on a mesoporous silica protocell core (e.g., core 110) as measured by a Zeta potential may varied monotonically from −50 millivolts (mV) to +50 MV by modification with an amine silane such as 2-(aminoethyl) propyltrimethoxy-silane (AEPTMS) or other organosilanes. This charge modification may affect the loading of a cargo into the protocell. Generally, after fusion of a lipid bilayer (lipid bilayer 120), a Zeta potential is reduced to between about −10 mV to +5 mV.

Referring again to FIG. 7 and quadrant 140 of protocell 100, in one embodiment, the one or more lipid bilayer 145 is modified with ligands that promote uptake by antigen-presenting cells (APCs). To generate antigen-loaded MANPs, APTES or HMDS-modified particles can be soaked in an aqueous solution of the desired antigen(s) for four hours at 4° C., remove unencapsulated proteins via centrifugation, and fuse liposomes to cargo-loaded cores as previously described; MANPs with a low degree of framework condensation will be used in formulations with supported lipid bilayer (SLB) 145 to ensure rapid antigen release upon APC uptake. To promote uptake by APCs, SLB 145 can be modified with 5 wt % of a DEC-205 scFv (prepared according to Johnson, et al.) (reference numeral 146), 5 wt % of human complement C3b (reference numeral 147) (binds to human and mouse CR1 and is commercially available from EMD Millipore), or 30 wt % of mannosylated cholesterol (reference numeral 148) (prepared according to Kawakami, et al.). Sulfo-SMCC can be employ to cross-link scFvs with a C-terminal cysteine residue to SLBs composed of DOPC with 5 wt % of 1,2-dioleoyl-sn-glycero-3-phosphoethanolamine (DOPE) and 10 wt % of 1,2-dioleoyl-sn-glycero-3-phosphoethanolamine-N-[methoxy(polyethylene glycol)-2000] (18:1 PEG-2000 PE). In one embodiment, SLBs composed of the cationic lipid, 1,2-dioleoyl-3-trimethylammonium-propane (DOTAP) can be used to promote adsorption of complement proteins. SLBs modified with mannosylated cholesterol can be formed by lyophilizing 60 wt % of DOPC, 10 wt % of 18:1 PEG-2000 PE, and 30 wt % of mannosylated cholesterol together, prior to rehydration of the lipid film and extrusion of resulting liposomes. Finally, all APC-targeted MANPs can be modified with H5WYG endosomolytic peptide 149. The quantity of adsorbed, cross-linked, and encapsulated antigen can be determined using a NanoDrop spectrophotometer. FIG. 7 is directed at protocells including a MANP core loaded with a cargo of a protein antigen. In another embodiment, the core is a mesoparticle silica nanoparticle (MSNP).

Once bound and taken up by the target cells, the loaded protocells can release cargo components from the porous particle and transport the released cargo components into the target cell. For example, sealed within the protocell by the liposome fused bilayer on the porous particle core, the cargo components can be released from the pores of the lipid bilayer, transported across the protocell membrane of the lipid bilayer and delivered within the targeted cell. In embodiments, the cargo release can be determined by, for example, interactions between the porous core and the lipid bilayer and/or other parameters such as pH value of the system. For example, the release of cargo can be achieved through the lipid bilayer, through dissolution of the porous silica; while the release of the cargo from the protocells can be pH-dependent.

In addition to targeted release of cargo from protocells, in another embodiment, a systemic release is contemplated.

In certain embodiments, the pH value for cargo is often less than 7, preferably about 4.5 to about 6.0, but can be about pH 14 or less. Lower pHs tend to facilitate the release of the cargo components significantly more than compared with high pHs. Lower pHs tend to be advantageous because the endosomal compartments inside most cells are at low pHs (about 5.5), but the rate of delivery of cargo at the cell can be influenced by the pH of the cargo. Depending upon the cargo and the pH at which the cargo is released from the protocell, the release of cargo can be relatively short (a few hours to a day or so) or a span for several days to about 20-30 days or longer. Thus, the embodiments may accommodate immediate release and/or sustained release applications from the protocells themselves.

In certain embodiments, the inclusion of surfactants can be provided to rapidly rupture the lipid bilayer, transporting the cargo components across the lipid bilayer of the protocell as well as the targeted cell. In certain embodiments, the phospholipid bilayer of the protocells can be ruptured by the application/release of a surfactant such as sodium dodecyl sulfate (SDS), among others to facilitate a rapid release of cargo from the protocell into the targeted cell or systematically. In certain embodiments, the rupture of the lipid bilayer can in turn induce immediate and complete release of the cargo components from the pores of the particle core of the protocells. In this manner, the protocell platform can provide versatile delivery systems as compared with other delivery systems in the art. For example, when compared to delivery systems using nanoparticles only, the disclosed protocell platform can provide a simple system and can take advantage of the low toxicity and immuneity of liposomes or lipid bilayers along with their ability to be PEGylated or to be conjugated to extend circulation time and effect targeting. In another example, when compared to delivery systems using liposome only, the protocell platform can provide a more stable system and can take advantage of the mesoporous core to control the loading and/or release profile.

In addition, the lipid bilayer and its fusion on porous particle core can be fine-tuned to control the loading, release, and targeting profiles and can further comprise fusogenic peptides and related peptides to facilitate delivery of the protocells for greater therapeutic effect.

Pharmaceutical compositions according to the present invention comprise an effective population of MANPS protocells as otherwise described herein formulated to effect an intended result (e.g. immune result, therapeutic result and/or diagnostic analysis, including the monitoring of therapy) formulated in combination with a pharmaceutically acceptable carrier, additive or excipient. The MANPS protocells within the population of the composition may be the same or different depending upon the desired result to be obtained. Pharmaceutical compositions according to the present invention may also comprise an addition bioactive agent or drug, such as an antibiotic or antiviral agent.

Generally, dosages and routes of administration of the compound are determined according to the size and condition of the subject, according to standard pharmaceutical practices. Dose levels employed can vary widely, and can readily be determined by those of skill in the art. Typically, amounts in the milligram up to gram quantities are employed. The composition may be administered to a subject by various routes, e.g. orally, transdermally, perineurally or parenterally, that is, by intravenous, subcutaneous, intraperitoneal, intrathecal or intramuscular injection, among others, including buccal, rectal and transdermal administration. Subjects contemplated for treatment according to the method of the invention include humans, companion animals, laboratory animals, and the like. The invention contemplates immediate and/or sustained/controlled release compositions, including compositions which comprise both immediate and sustained release formulations. This is particularly true when different populations of protocells are used in the pharmaceutical compositions or when additional bioactive agent(s) are used in combination with one or more populations of protocells as otherwise described herein.

Formulations containing the compounds according to the present invention may take the form of liquid, solid, semi-solid or lyophilized powder forms, such as, for example, solutions, suspensions, emulsions, sustained-release formulations, tablets, capsules, powders, suppositories, creams, ointments, lotions, aerosols, patches or the like, preferably in unit dosage forms suitable for simple administration of precise dosages.

Pharmaceutical compositions according to the present invention typically include a conventional pharmaceutical carrier or excipient and may additionally include other medicinal agents, carriers, adjuvants, additives and the like. Preferably, the composition is about 0.1% to about 85%, about 0.5% to about 75% by weight of a compound or compounds of the invention, with the remainder consisting essentially of suitable pharmaceutical excipients.

An injectable composition for parenteral administration (e.g. intravenous, intramuscular or intrathecal) will typically contain the compound in a suitable i.v. solution, such as sterile physiological salt solution. The composition may also be formulated as a suspension in an aqueous emulsion.

Liquid compositions can be prepared by dissolving or dispersing the population of protoells (about 0.5% to about 20% by weight or more), and optional pharmaceutical adjuvants, in a carrier, such as, for example, aqueous saline, aqueous dextrose, glycerol, or ethanol, to form a solution or suspension. For use in an oral liquid preparation, the composition may be prepared as a solution, suspension, emulsion, or syrup, being supplied either in liquid form or a dried form suitable for hydration in water or normal saline.

For oral administration, such excipients include pharmaceutical grades of mannitol, lactose, starch, magnesium stearate, sodium saccharine, talcum, cellulose, glucose, gelatin, sucrose, magnesium carbonate, and the like. If desired, the composition may also contain minor amounts of non-toxic auxiliary substances such as wetting agents, emulsifying agents, or buffers.

When the composition is employed in the form of solid preparations for oral administration, the preparations may be tablets, granules, powders, capsules or the like. In a tablet formulation, the composition is typically formulated with additives, e.g. an excipient such as a saccharide or cellulose preparation, a binder such as starch paste or methyl cellulose, a filler, a disintegrator, and other additives typically used in the manufacture of medical preparations.

Methods for preparing such dosage forms are known or is apparent to those skilled in the art; for example, see Remington's Pharmaceutical Sciences (17th Ed., Mack Pub. Co., 1985). The composition to be administered will contain a quantity of the selected compound in a pharmaceutically effective amount for therapeutic use in a biological system, including a patient or subject according to the present invention.

Methods of treating patients or subjects in need for a particular disease state or infection comprise administration an effective amount of a pharmaceutical composition comprising therapeutic MANPS protocells and optionally at least one additional bioactive (e.g. antiviral) agent according to the present invention.

Diagnostic methods according to the present invention comprise administering to a patient in need an effective amount of a population of diagnostic MANPS protocells (e.g., protocells which comprise a target species, such as a targeting peptide which binds selectively to cancer cells and a reporter component to indicate the binding of the MANPS protocells whereupon the binding of protocells to cells as evidenced by the reporter component (moiety) will enable a diagnosis of the existence of a disease state in the patient.

An alternative of the diagnostic method of the present invention can be used to monitor the therapy of a disease state in a patient, the method comprising administering an effective population of diagnostic MANHPS protocells (e.g., protocells which comprise a target species, such as a targeting peptide which binds selectively to target cells and a reporter component to indicate the binding of the protocells to cancer cells if the cancer cells are present) to a patient or subject prior to treatment, determining the level of binding of diagnostic protocells to target cells in said patient and during and/or after therapy, determining the level of binding of diagnostic protocells to target cells in said patient, whereupon the difference in binding before the start of therapy in the patient and during and/or after therapy will evidence the effectiveness of therapy in the patient, including whether the patient has completed therapy or whether the disease state has been inhibited or eliminated (including elimination of an infectious disease state or remission of a cancer).

The following non-limiting examples are illustrative of the invention and its advantageous properties, and are not to be taken as limiting the disclosure or claims in any way. In the examples, as well as elsewhere in this application, all parts and percentages are by weight unless otherwise indicated.

### Example 1

### Mesoporous Nanoparticles with Reproducible Properties can be Synthesized in a Scalable Fashion Via Aerosol-Assisted Evaporation-Induced Self-Assembly

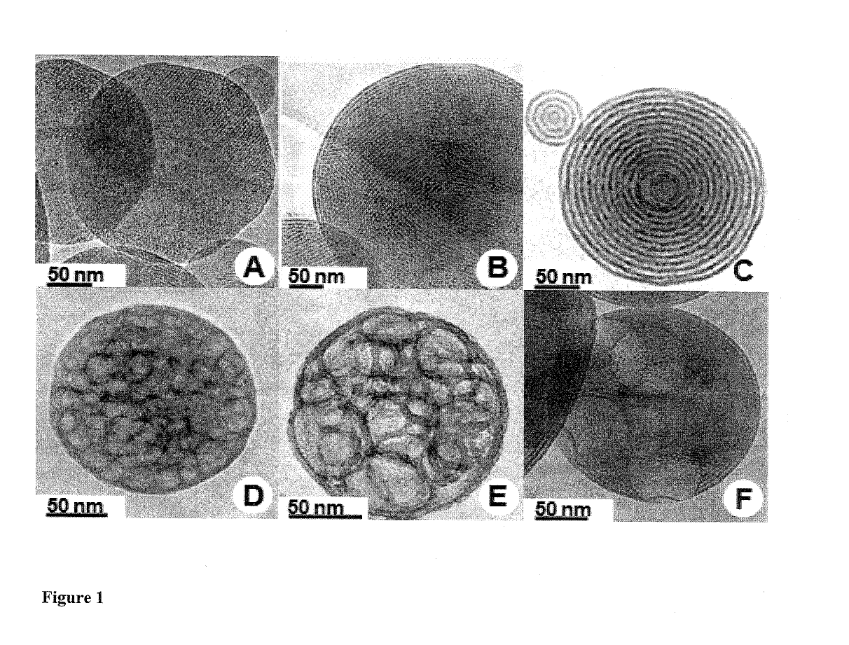

Aerosol-assisted evaporation-induced self-assembly (EISA)⁶ is a robust, scalable process that we pioneered over a decade ago to synthesize spherical, well-ordered oxide nano- and microparticles with a variety of pore sizes and geometries (see FIG. 1).

Panels (A) (B) of FIG. 1 show electron microscopy images of MSNPs with 2.5-nm pores (A) or 25-nm pores (B). The inset in (B) demonstrates that pores are surface-accessible. (C) The loading capacities of MSNPs with 2.5-nm or 25-nm pores for E. coli O157:H7 lipopolysaccharide (LPS), anthrax protective antigen (PA), soluble Nipah virus glycoprotein (sG), ricin toxin A-chain (RTA), ovalbumin (OVA), and lysozyme (LSZ). MSNPs were modified with (3-aminopropyl)triethoxysilane (APTES) to make pores positively-charged and with hexamethyldisilazane (HMDS) to make pores more hydrophobic. Capacity scales roughly with size (LPS>PA>sG>OVA RTA>LSZ), charge (pI of LSZ ˜11 vs. pI of PA, sG, RTA, and OVA ˜4-5), and degree of hydrophobicity (log P ˜10 for LPS). Panel (D) shows the percentages of free OVA and OVA loaded in MSNPs (25-nm pores, modified with 10% APTES, encapsulated within a SLB composed of DOPC) that remain intact, i.e. recognizable by a conformational antibody, after storage in 1×PBS at 4° C. or at room temperature for the indicated periods of time. Release of OVA from MSNPs was triggered by digesting the SLB with lipase. For (C) and (D), data represent the mean±std. dev. for n=3.

In the aerosol-assisted EISA process, a dilute solution of a metal salt or metal alkoxide is dissolved in an alcohol/water solvent along with an amphiphilic structure-directing surfactant or block co-polymer; the resulting sol is then aerosolized with a carrier gas and introduced into a laminar flow reactor. Solvent evaporation drives a radially-directed self-assembly process to form particles with systematically variable pores sizes (2 to 50-nm), pore geometries (hexagonal, cubic, lamellar, cellular, etc.), and surface areas (100 to >1,200 m²/g). Aerosol-assisted EISA, additionally, produces particles compatible with a variety of post-synthesis processing procedures, enabling the hydrodynamic size to be varied from 30-nm to >10-μm and the pore walls to be modified with a wide range of functional moieties (e.g. primary amine groups) that facilitate selective crosslinking strategies. Although originally developed for the synthesis of so-called mesoporous silica nanoparticles (MSNPs), we have recently extended the aerosol-assisted EISA approach to the formation of mesoporous nanoparticles composed of aluminum hydroxide or sulfate, as described below.

In anticipation of manufacturing high-quality MANPs in the quantities necessary for use in humans, we have developed manufacturing and characterization processes with current good manufacturing practice (cGMP) principles in mind, as set forth by the US FDA under Section 501(B) of the 1938 Food, Drug, and Cosmetic Act (21 USCS §351). For example, our lab-scale procedure for generating nanoparticles via aerosol-assisted EISA is computer-controlled, well-documented, and has a minimal number of steps, each of which can be performed by low-skill operators after brief training. Each batch of nanoparticles is fully characterized, and the resulting information is batch-traceable and recorded both electronically and on paper. We, furthermore, characterize the physiochemical characteristics and in vitro behavior of resulting particles using a battery of tests developed by NIST and approved by the Nanotechnology Characterization Laboratory (NCL) for nanoparticles with potential human applications (see http://ncl.cancer.gov/working_assay-cascade.asp). To facilitate scale up from our current production rate of one gram/day, we have designed our lab-scale reactor as a computer-controlled, flow-through system that features a small footprint and requires minimal specialized equipment. With this design, addition of parallel units, all of which can be monitored and controlled by the original computer system, enables the process to be scaled to any desired quantity in a quick and cost-effective manner.

### Example 2

### Optimization of Pore Size and Chemistry Enables High Capacity Loading and Long-Term Stabilization of Disparate Protein Antigens, while Optimization of Framework Condensation Results in Tailorable Release Rates

Simple liposomes have a limited capacity for proteins >30 kDa and release encapsulated proteins within 12 to 72 hours, even when stabilized with polyethylene glycol (PEG) and cholesterol.⁷ Preparation of multilamellar vesicles (MLVs) using a dehydration-rehydration method for aqueous entrapment of macromolecules can increase encapsulation efficiency by as much as 50% but only minimally prolongs the duration of protein release.⁸ Polymeric nanoparticles, such as those composed of poly(lactic-co-glycolic acid) (PLGA) and prepared using a double-emulsion solvent-evaporation technique, have a 2 to 5-fold lower capacity for relatively small (<50 kDa), globular proteins than MLVs of the same approximate size; sustained release can be achieved, however, by crosslinking protein to the polymer framework.⁹ Despite these recent improvements, state-of-the-art MLVs and polymeric nanoparticles still suffer from several limitations, including complex processing techniques that are highly sensitive to pH, temperature, ionic strength, presence of organic solvents, lipid or polymer size and composition, and physicochemical properties of the cargo molecule, all of which impact the resulting nanoparticle's size, stability, entrapment efficiency, and release rate.¹⁰ In contrast, mesoporous oxide nanoparticles, such as MSNPs, have capacities for physicochemically disparate molecules that exceed those of liposomes and polymeric nanoparticles by 100 to 1,000-fold and can be easily engineered for burst or sustained release.^{7, 11}

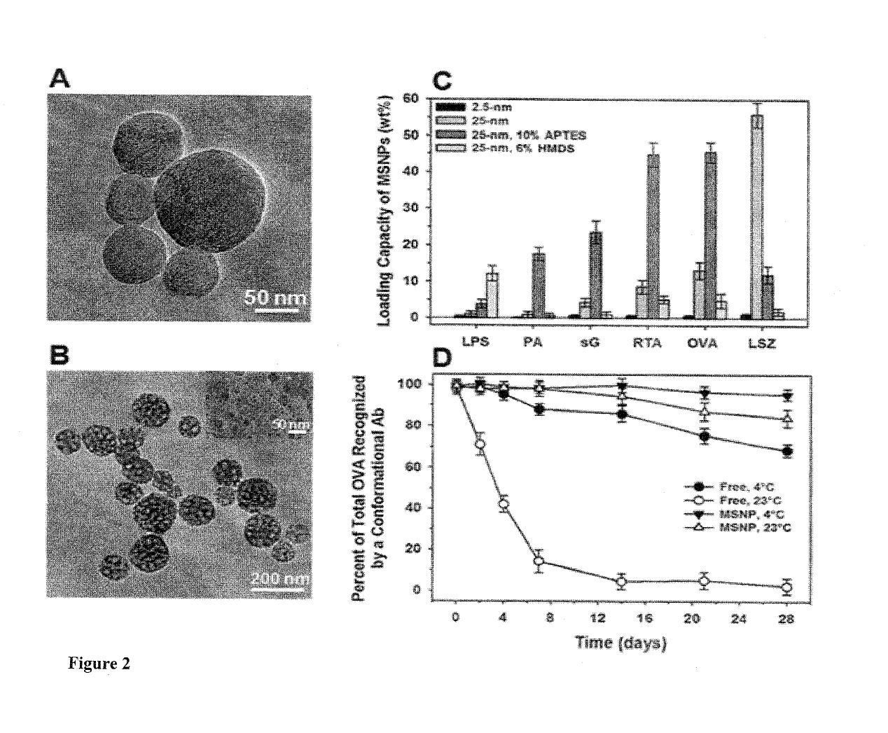

FIG. 2 illustrates that MSNPs have a high capacity for physicochemically disparate proteins and maintain long-term stability of encapsulated proteins in the absence of cold chain. Panels (A)-(B) of FIG. 2 show electron microscopy images of MSNPs with 2.5-nm pores (A) or 25-nm pores (B). The inset in (B) demonstrates that pores are surface-accessible. (C) The loading capacities of MSNPs with 2.5-nm or 25-nm pores for E. coli O157:H7 lipopolysaccharide (LPS), anthrax protective antigen (PA), soluble Nipah virus glycoprotein (sG), ricin toxin A-chain (RTA), ovalbumin (OVA), and lysozyme (LSZ). MSNPs were modified with (3-aminopropyl)triethoxysilane (APTES) to make pores positively-charged and with hexamethyldisilazane (HMDS) to make pores more hydrophobic. Capacity scales roughly with size (LPS>PA>sG>OVA˜RTA>LSZ), charge (pI of LSZ˜11 vs. pI of PA, sG, RTA, and OVA ˜4-5), and degree of hydrophobicity (log P ˜10 for LPS). Panel (D) illustrates the percentages of free OVA and OVA loaded in MSNPs (25-nm pores, modified with 10% APTES, encapsulated within a SLB composed of DOPC) that remain intact, i.e. recognizable by a conformational antibody, after storage in 1×PBS at 4° C. or at room temperature for the indicated periods of time. Release of OVA from MSNPs was triggered by digesting the SLB with lipase. For (C) and (D), data represent the mean±std. dev. for n=3.

As demonstrated by FIG. 2C, the pore size and chemistry of MSNPs can be modulated to promote high capacity loading (10-50 wt %) for a wide variety of proteins using a simple loading procedure that is universally applicable to small molecule drugs, RNA, DNA, and proteins.¹¹ MSNPs also stabilize encapsulated proteins and enable long-term (>1 month), room-temperature storage (see FIG. 2D), which can be further enhanced when particles are lyopholized rather than being maintained in liquid media (data not shown). MSNPs, additionally, have tailorable release rates, which can be modulated by varying the degree to which the silica framework is condensed and, therefore, the rate of its dissolution via hydrolysis under physiological conditions.¹¹

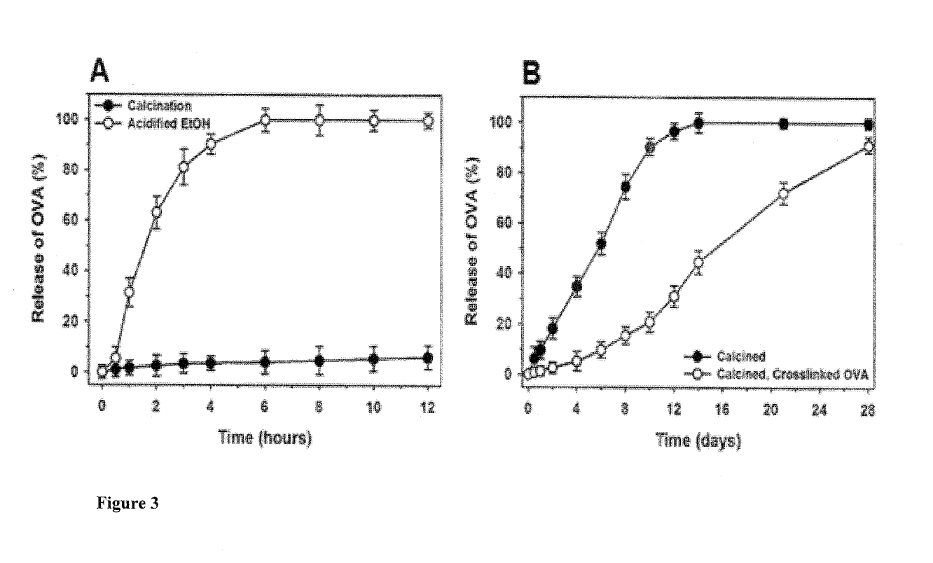

FIG. 3 illustrates the degree of condensation of the MSNP framework can be optimized for burst or sustained release of encapsulated OVA. Panels (A)-(B) of FIG. 3 show the percentage of OVA released from MSNPs with a low (‘Acidified EtOH’) or high (‘Calcination’) degree of framework condensation upon incubation in a simulated body fluid (10% serum, pH 7.4) at 37° C. for the indicated periods of time. A low degree of silica condensation was achieved using acidified ethanol (EtOH) to extract structure-directing surfactants, while a high degree of silica condensation was promoted via thermal calcination; release of OVA from calcined MSNPs was further delayed by crosslinking the protein to the APTES-modified pore network using 1-ethyl-3-[3-dimethylaminopropyl]carbodiimide hydrochloride (EDC). Data represent the mean±std. dev. for n=3

As shown in FIG. 3A, MSNPs with a low degree of silica condensation release encapsulated ovalbumin (OVA) within ˜6 hours, while MSNPs with a high degree of silica condensation release encapsulated OVA over a period of 10-14 days. Release rates can be further diminished through chemical conjugation of the protein to the pore network using a variety of well-established chemistries (see FIG. 3B).¹²

### Example 3

### Modification of the Supported Lipid Bilayer with Various Targeting Ligands Promotes Efficient Uptake by Antigen-Presenting Cells

A number of factors govern uptake and processing of nanoparticles by APCs, including their size, shape, surface charge, and degree of hydrophobicity. ^{1, 13, 14} Furthermore, a variety of molecules have been employed to target nanoparticles to APCs, including those that bind to CD205 (a.k.a. DEC-205), the mannose receptor (a.k.a. CD206), CD11b/CD18 (a.k.a. CR3), Fcγ receptors, and various TLRs.^{1 14, 15} To promote uptake by APCs, we encapsulated cargo-loaded MSNPs within SLBs composed of 1,2-dioleoyl-sn-glycerol-3-phosphocholine (DOPC), which we further modified with human IgG, human complement (C3), mannosylated cholesterol,¹⁶ or the TLR-4 agonist, monophosphoryl lipid A (MPLA).

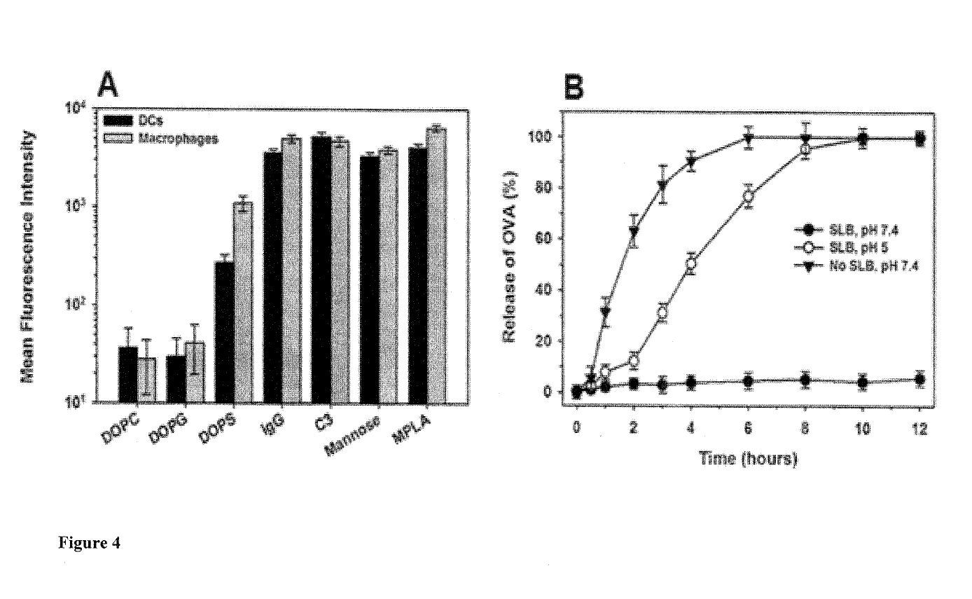

FIG. 4 illustrates the encapsulation of OVA-loaded MSNPs in a SLB that is further modified with targeting ligands enables efficient uptake by dendritic cells and macrophages and pH-triggered release of OVA. Panel (A) shows mean fluorescence intensities of 1×10⁶ human dendritic cells (DCs) and macrophages after incubation with a 10⁴-fold excess of MSNPs for 1 hour at 37° C. MSNPs were encapsulated in DOPC SLBs modified with 5 wt % of human IgG, 5 wt % of human complement C3, 30 wt % of mannosylated cholesterol, or 5 wt % of MPLA; MSNPs encapsulated in SLBs composed of DOPS or DOPG were included as controls. MSNPs were labeled with pHrodo Red, the fluorescence intensity of which dramatically increases under acidic (i.e. phagosomal or endosomal) conditions. Panel (B) shows the percentage of OVA released from MSNPs encapsulated in DOPC SLBs upon incubation in a simulated body fluid (10% serum, pH 7.4) or a pH 5.0 buffer at 37° C. for the indicated periods of time. The OVA release profile for MSNPs without a SLB is included for comparison. Surfactants were extracted using acidified ethanol, resulting in a low degree of silica condensation. Data represent the mean±std. dev. for n=3.

As shown in FIG. 4A, all of the aforementioned ligands induce efficient uptake of MSNPs by human dendritic cells (DCs)¹⁷ and macrophages¹⁸ derived from peripheral blood monocytes; macrophages also internalize MSNPs coated with 1,2-dioleoyl-sn-glycero-3-phospho-L-serine (DOPS) but not MSNPs coated with other negatively-charged phospholipids (e.g. 1,2-dioleoyl-sn-glycero-3-phospho-(1′-rac-glycerol), or DOPG), possibly due to the action of scavenger receptors.¹ In addition to providing a fluid interface for display of targeting moieties,¹¹ the SLB also enables stable retention of cargos under neutral pH conditions and triggered release of cargo under acidic pH (i.e. endosomal or phagosomal) conditions (see FIG. 4B).

### Example 4

### Mesoporous Oxide Nanoparticles Loaded with a Model Antigen Induce Humoral and Cellular Responses when Targeted to APCs

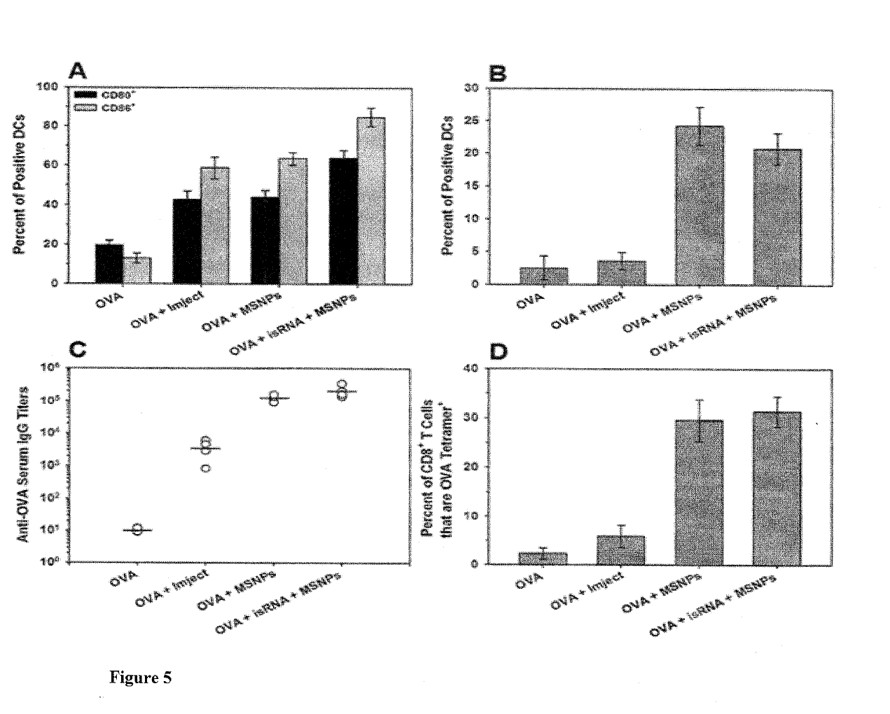

Engineered nano- and microparticles that co-deliver antigen and immunostimulatory molecules are of great interest as next-generation subunit vaccines and so-called ‘smart’ adjuvants, given their ability to mimic viruses and bacteria while avoiding toxicity and anti-vector immune responses.¹⁴ To demonstrate that mesoporous oxide nanoparticles warrant development as particulate vaccines and adjuvants, we co-loaded MSNPs with a model protein antigen (OVA) and an immunostimulatory RNA (isRNA) known to activate TLR7 and TLR8¹⁹ and then encapsulated cargo-loaded MSNPs in a SLB that we further modified with targeting and endosomolytic moieties. We found that high-surface-area MSNPs were able to encapsulate 50-60 wt % of OVA or isRNA individually and simultaneously encapsulate ˜30 wt % of both OVA and isRNA, capacities that exceed those of state-of the-art liposomes and polymeric nanoparticles by 2 to 100-fold.⁵ Furthermore, as described above, a SLB composition of DOPC modified with 5 wt % of MPLA triggered efficient uptake of MSNPs by human dendritic cells (DCs) derived from peripheral blood monocytes. We have previously shown that endolysosome acidification destabilizes the SLB, thereby exposing the MSNP core and stimulating its dissolution; we have also shown that incorporating endosomolytic peptides (e.g. ‘H5WYG’²⁰) on the SLB promotes release of MSNP-encapsulated cargo in the cytosol of target cells. ^{7, 11, 21} These phenomena enable endosomal release of isRNA and cytosolic dispersion of OVA, which, in turn, trigger DC maturation and cross-presentation of OVA-derived peptides, as demonstrated by FIG. 5.

FIG. 5 shows in vitro and in vivo assessment of MPLA-targeted, OVA-loaded MSNPs in the absence and presence of isRNA. (A)-(B) Human DCs isolated from peripheral blood monocytes were incubated with 1 μg/mL of free OVA or equivalent doses of OVA complexed to Imject® Alum or loaded in MSNPs for 24 hours. DCs were then probed with FITC-labeled monoclonal antibodies against CD80 (A), CD86 (A), or the OVA-derived peptide, SIINFEKL complexed with MHC class I H-2K^{b} molecules (B). (C)-(D) Groups of four C57B1/6 mice were immunized intramuscularly on days 0, 14, 28, and 42 with 10 μg of OVA in free, Imject® Alum, or MSNP formulations. Sera from immunized mice was collected on day 56 and analyzed by ELISA for OVA-specific IgG (C) and by flow cytometry for the percentage of PBMCs double-positive for CD8 and an OVA peptide-MHC tetramer (D). MPLA and isRNA doses delivered via MSNPs were ˜0.1 μg/mL for (A)-(B) and ˜1 μg for (C)-(D). Data represent the mean std. dev. for n=3.

In accordance with previous studies,¹³ we found that isRNA triggers increased DC maturation (see FIG. 5A) but has no significant effect on cross-presentation (see FIG. 5B). In contrast, OVA-loaded MSNPs, in both the absence and presence of isRNA, enhance antigen cross-presentation (see FIG. 5B), as is expected for particulate antigen delivery systems.⁵ MPLA-targeted, OVA-loaded MSNPs, additionally, induce high-titer, OVA-specific antibody responses (see FIG. 5C) and elicit OVA-specific CD8T cell responses (see FIG. 5D) upon immunization of C57B1/6 mice, indicating that mesoporous oxide nanoparticles are an important class of antigen delivery vehicles and warrant further development. In the proposed work, we seek to further enhance the efficacy of APC-targeted, antigen-loaded mesoporous oxide nanoparticles by utilizing mesoporous alum for antigen adsorption, presentation, and delivery.

### Example 5

### Synthesis and Characterization of MANPs with Appropriate Particle Sizes, Pore Sizes, and Dissolution Rates

Aluminum chloride (AlCl₃.6H₂O) and alum (KAKSO₄)₂.12H₂O) are used as precursors to synthesize aluminum hydroxide and aluminum sulfate nanoparticles, respectively. These precursors are dissolved in an alcohol/water solvent along with ionic or non-ionic surfactants, block co-polymers, or polymeric templates and aerosol processed as described by Jung, et al.²² Surfactants/templates are extracted using either acidified ethanol or thermal calcination to yield mesoporous hydroxides (boehmite AlO(OH) or gibbsite Al(OH)₃) or sulfates (alum); mesoporous alum nanoparticles are used for all further studies unless mesoporous aluminum hydroxide nanoparticles prove to have superior properties or manufacturability. To enable facile adsorption of a single antigen or cocktails of multiple antigens, initially synthesize MANPs with a surface area of ˜500 m²/g and 10-nm pores templated by Pluronic P123;²³ if necessary, increase the average pore size to accommodate larger antigens (e.g. F. tularensis lipopolysaccharide). Pore size and surface area is determined by N₂ sorption porosimetry, and particle size distributions is measured using Dynamic Light Scattering (DLS) and Transmission Electron Microscopy (TEM). Center particle size distributions at 100-nm by solution concentration or filtration, as previously described.¹¹

### Example 6

### Modifying MANP Surfaces with Cationic or Hydrophobic Moieties to Facilitate Antigen Loading

Given that the MANPs described herein are naturally negatively-charged (ζ=−20 mV in 0.5×PBS, pH 7.4), soak them in a 10 mol % solution of the amine-containing silane, (3-aminopropyl)triethoxysilane (APTES) for 6 hours at room temperature. The pore network of resulting particles will contain primary amine groups and should, therefore, readily adsorb the majority of the antigens described in subtask 4.3.1. To facilitate absorption of amphiphilic antigens (e.g. F. tularensis lipopolysaccharide), modify MANPs with hexamethyldisilazane (HMDS) by soaking them in a 6 mol % solution for 6 hours at room temperature. To facilitate antigen crosslinking, soak MANPs in a 5 mol % solution of (3-mercaptopropyl)trimethoxysilane (MPTS) for 2 hours at room temperature. Determine the overall zeta potential of APTES, HMDS, and MPTS-modified MANPs and quantify the approximate density of primary amine (—NH₃), methyl (—CH₃), and sulfhydryl (—SH) moieties using Fourier transform infrared (FTIR) spectroscopy. MANPs with the properties necessary to enable high capacity loading of physicochemically disparate antigens are thus provided.

### Example 7

### Loading of Mesoporous Alum Nanoparticles with Model Antigens and Assessment of Colloidal Stability and Antigen Release Kinetics in Simulated Body Fluids

Antigens Isolated from Model Category A and B Biothreat Agents.

To demonstrate that MANPs can be loaded with a variety of antigens, procure various proteins, including glycoproteins and lipoproteins, derived from Category A or B bacteria, viruses, and toxins. Purchase lipopolysaccharide (LPS) isolated from the live vaccine strain (LVS) of F. tularensis (Ft), subsp. holarctica from BEI Resources, recombinant Bacillus anthracis protective antigen (PA) from EMD Millipore, recombinant botulinum neurotoxin type A (BoNT-A) light chain (LC) from R&D Systems, and deglycosylated ricin toxin A-chain (RTA) from Sigma-Aldrich. Have recombinant Ft intracellular growth locus C (Ig1C) with a C-terminal (His)₆ affinity tag custom synthesized by Proteos, Inc. Produce soluble Zaire Ebola virus glycoprotein (sGP) and soluble GP1 (sGP1) derived from the Lassa fever virus GPC gene using a technique similar to the procedure used by Negrete, et al. for the production of soluble Nipah virus glycoprotein.²⁴ Briefly, full-length Ebola GP are cloned in-frame with an N-terminal (His)₆ affinity tag; a similar construct has been shown to undergo the complex post-translational modification of native GP, including furin cleavage and homotrimer formation.²⁵ A stable sGP-secreting cell line is generated by transfecting sGP plasmid into human 293F cells and selecting for clones using antibiotics, followed by limiting dilution cloning. sGP is prepared by growing cells in shaker cultures using serum-free medium and purified by nickel affinity and size exclusion chromatography. Produce the full ectodomain of Lassa GP1 with a C-terminal (His)₆ tag and mutate the SKI-1/S1P protease recognition domain at the C-terminus of GP1 from RRLL to RRAA to abrogate cleavage of the downstream purification tag. This construct will be expressed in a mammalian cell system using reported methodologies in order to preserve native glycosylation patterns.²⁶

Choice of Antigens.

The aforementioned antigens are selected since they encompass a range of molecular weights (10-100 kDa), isoelectric points (pI=4-6), and degrees of hydrophobicity (log P=−1-+10) and will, therefore, help demonstrate that MANPs can adsorb and deliver physicochemically disparate molecules. The Ft pathogenicity island protein, Ig1C, is selected as a model T cell antigen because mice,²⁷ rats (unpublished data), and non-human primates (unpublished data) immunized with a live, attenuated Listeria monocytogenes vaccine expressing Ig1C developed partial to full protection against respiratory challenge with the highly virulent Ft SCHU S4 astrain; this vaccine can be optimized against aerosol SCHU S4 challenge in rats and non-human primates as part of a DTRA-funded project (HDTRA1-12-C-0046) and it can be determined whether the MANP-based adjuvants can induce antibody responses against Ig1C to complement this contract. Ft LPS is chosen as a model B cell antigen because it is thought to be a protective antigen in mice²⁸⁻³⁰ and humans; although the role of antibodies in protection against Ft has been controversial, recent studies in our lab and others have clearly demonstrated that antibodies increase resistance against SCHU S4 challenge.³¹ Select anthrax PA since it, in combination with various adjuvants, has been proven to protect guinea pigs against challenge with virulent B. anthracis spores.³² Choose Ebola sGP and Lassa sGP1 as model filo- and arenavirus antigens since a recent report indicated that a sGP subunit vaccine confers protection against lethal Ebola virus challenge when the protein is produced in a mammalian expression system, a result that should be applicable to arenaviruses as well.²⁵ Finally, vaccines composed of recombinant RTA and polypeptides derived from BoNT serotypes A, B, and E have been shown to protect mice from challenge with lethal doses of ricin or botulinum toxins;^{33, 34} choose to use toxin fragments for safety and regulatory reasons.

### Example 8

### Synthesis of Antigen-Loaded MANPs and Quantify Loading Capacities

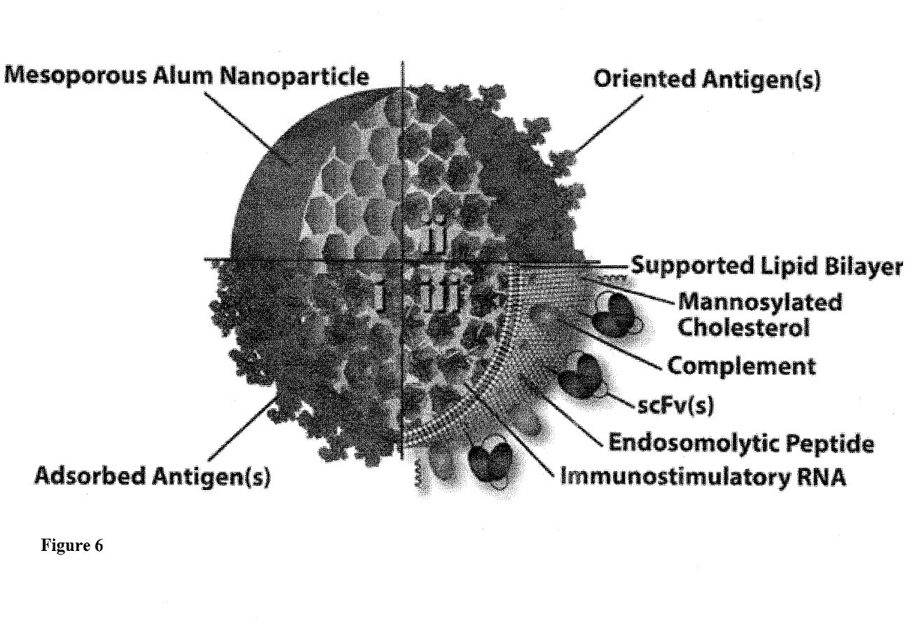

As depicted in FIG. 6, prepare three general formulations: (1) MANPs with randomly-adsorbed antigens, (2) MANPs to which antigens have been crosslinked to facilitate antigen orientation and high density presentation, and (3) antigen-loaded MANPs encapsulated in SLBs modified with ligands that promote uptake by APCs. Utilize APTES-modified MANPs for random adsorption of Ig1C, PA, sGP, sGP1, RTA, and BoNT-A LC and HMDS-modified MANPs for random adsorption of LPS; also co-load Ig1C and LPS using MANPs modified with both APTES and HMDS. To promote antigen adsorption, MANPs are soaked in an aqueous solution of the desired antigen(s) for 12 hours at 4° C. and washed three times with 1×PBS to remove unencapsulated antigen. MANPs with a high degree of framework condensation will be used for random adsorption of antigen(s) since resulting particles will likely act as an antigen depot and should, therefore, release antigen over a period of 1-2 weeks to maximize interaction times between antigen and APCs.

To crosslink Ig1C, PA, sGP, sGP1, RTA, and BoNT-A LC to MANPs, incubate MPTS-modified particles with a 10-fold molar excess of the non-cleavable amine-to-sulfhydryl crosslinker, sulfosuccinimidyl-4-(N-maleimidomethyl)cyclohexane-1-carboxylate (sulfo-SMCC) or the reducible amine-to-sulfhydryl crosslinker, sulfosuccinimidyl 6-(3′-[2-pyridyldithio]-propionamido)hexanoate (sulfo-LC-SPDP) for 1 hour at room temperature; crosslinker-activated particles will then be incubated with 1 mg/mL of antigen overnight at 4° C. To crosslink LPS, employ the sulfhydryl-to-hydroxyl crosslinker, N-(p-maleimidiophenyl)isocyanate (PMPI). The pore network with Ni(II) complexes¹² can also be modified to orient and immobilize proteins with (His)₆ affinity tags. Although it is anticipated that a high density of surface-exposed antigen will trigger maximal uptake by APCs, if necessary, vary the reaction stoichiometry to control the density of crosslinked antigens. MANPs with a high degree of framework condensation will be used in formulations with crosslinked antigen(s).

To generate antigen-loaded MANPs, soak APTES or HMDS-modified particles in an aqueous solution of the desired antigen(s) for 4 hours at 4° C., remove unencapsulated proteins via centrifugation, and fuse liposomes to cargo-loaded cores as previously described;^{7, 11, 21} MANPs with a low degree of framework condensation are used in formulations with a SLB to ensure rapid antigen release upon APC uptake. To promote uptake by APCs, modify the SLB with 5 wt % of a DEC-205 scFv (prepared according to Johnson, et al.³⁵), 5 wt % of human complement C3b (binds to human and mouse CR1 and is commercially available from EMD Millipore), or 30 wt % of mannosylated cholesterol (prepared according to Kawakami, et al.¹⁶). Employ sulfo-SMCC to crosslink scFvs with a C-terminal cysteine residue to SLBs composed of DOPC with 5 wt % of 1,2-dioleoyl-sn-glycero-3-phosphoethanolamine (DOPE) and 10 wt % of 1,2-dioleoyl-sn-glycero-3-phosphoethanolamine-N-[methoxy(polyethylene glycol)-2000] (18:1 PEG-2000 PE). Use SLBs composed of the cationic lipid, 1,2-dioleoyl-3-trimethylammonium-propane (DOTAP) to promote adsorption of complement proteins. SLBs modified with mannosylated cholesterol are formed by lyophilizing 60 wt % of DOPC, 10 wt % of 18:1 PEG-2000 PE, and 30 wt % of mannosylated cholesterol together, prior to rehydration of the lipid film and extrusion of resulting liposomes. Finally, modify all APC-targeted MANPs with the H5WYG endosomolytic peptide, as previously described.¹¹ Determine the quantity of adsorbed, crosslinked, and encapsulated antigen using a NanoDrop spectrophotometer.

In vitro and in vivo immuneity analyses can begin with MANPs loaded with Ft Ig1C and/or LPS as model T and B cell antigens, respectively; Table 1 provides a summary of antigen formulations that can be tested. Assess the colloidal stability and antigen-release characteristics of MANPs loaded with each of the seven model antigens, as well as a mixture of Ig1C and LPS.

TABLE 1
Antigen formulations that are tested in accordance with the experiment(s)
of Example 8. Eight antigen-loaded MANP formulations. Free antigen,
antigen adsorbed to Imject ® Alum, empty MANPs, and empty
MSNPs used as controls.

| | | Framework | Pore | | Loading | | |
|---|---|---|---|---|---|---|---|
| | Adjuvant | Condensation | Chemistry | Antigen(s) | Strategy | SLB | Ligand |
| 1 | MANP | High | NH₃ | IglC | Adsorbed | — | — |
| 2 | MANP | High | SH | IglC | Crosslinked | — | — |
| 3 | MANP | Low | NH₃ | IglC | Encapsulated | DOPC | DEC-205 scFv |
| 4 | MANP | High | CH₃ | LPS | Adsorbed | — | — |
| 5 | MANP | High | SH | LPS | Crosslinked | — | — |
| 6 | MANP | Low | CH₃ | LPS | Encapsulated | DOPC | DEC-205 scFv |
| 7 | MANP | High | NH₃/CH₃ | IglC/LPS | Adsorbed | — | — |
| 8 | MANP | Low | NH₃/CH₃ | IglC/LPS | Encapsulated | DOPC | DEC-205 scFv |
| 9 | — | — | — | IglC | — | — | — |
| 10 | — | — | — | LPS | — | — | — |
| 11 | — | — | — | IglC/LPS | — | — | — |
| 12 | Imject | — | — | IglC | Adsorbed | — | — |
| 13 | Imject | — | — | LPS | Adsorbed | — | — |
| 14 | Imject | — | — | IglC/LPS | Adsorbed | — | — |
| 15 | MANP | High | NH₃ | — | — | — | — |
| 16 | MSNP | High | NH₃ | — | — | — | — |

Since traditional alum adjuvants have been postulated to have a toxic effect on APCs,³⁷ monitor BMDC viability in each phagocytosis assay by including 7-aminoactinomycin D (7-AAD) to detect dead cells via flow cytometry. Assess the effect of MANPs and Imject® Alum on BMDC apoptosis over a period of 72 hours. To do so, BMDCs are incubated with the antigen formulations listed in Table 1 for two hours at 37° C., washed to remove extracellular antigen, and stained with FITC-labeled annexin V and propidium iodide (PI) immediately, as well as 12, 24, 48, and 72 hours post-exposure to assess cellular lysis and apoptosis.³⁸ Staining with FITC-labeled annexin V alone is indicative of the early stages of apoptosis, while staining with PI indicates a loss of membrane integrity associated with either the late stages of apoptosis or necrosis. Loss of viability immediately following MANP or Imject® Alum uptake will indicate that the formulation has a toxic effect. Apoptosis that occurs after the 24-hour time point may be due to toxicity of the antigen formulation or attributable to BMDC maturation and cytokine release,³⁸ both of which will be further evaluated as described herein.

### Example 9

### Assessment of the Colloidal Stability of Antigen-Loaded MANPs

To assess colloidal stability, determine the time-dependent particle size distribution of each MANP formulation upon incubation in a simulated body fluid (10% serum in phenol red-free Dulbecco's Modified Eagle Medium (DMEM), pH 7.4) at 37° C. Modify the surfaces of MANPs with randomly adsorbed or chemically crosslinked antigens with PEG-2000 to enhance colloidal stability if DLS reveals evidence of particle aggregation.

Quantify the Release Kinetics of Antigen-Loaded MANPs in Simulated Body Fluids.

To determine the rate of antigen release for each MANP formulation, incubate particles in a simulated body fluid (10% serum in phenol red-free DMEM, pH 7.4) at 37° C.; at appropriate time intervals (every 1-2 hours for particles with low framework condensation vs. every 1-2 days for particles with high framework condensation), pellet MANPs via centrifugation and assess the concentration of antigen in the supernatant as previously described.¹¹ Normalize resulting concentrations against the loading capacity for each MANP formulation to generate release profiles similar to those depicted in FIG. 3. Eight antigen-loaded MANP formulations for further characterization are thus prepared, and proof-of-concept data demonstrates that MANPs can be readily loaded with seven physicochemically disparate antigens isolated from potential biothreat agents.

### Example 10

### Evaluation of In Vitro Maturation of Dendritic Cells and Proliferation of Antigen-Specific CD8T Cells

Assess Uptake of Antigen-Loaded MANPs by Mouse Bone Marrow-Derived Dendritic Cells (BMDCs) and Effects on BMDC Viability.

Use flow cytometry and confocal microscopy to compare phagocytosis of MANPs with surface-adsorbed or surface-oriented antigens to MANPs encapsulated within SLBs modified with a DEC-205 scFv and the H5WYG endosomolytic peptide. Fluorescently-labeled MANPs and MSNPs (components of formulations 1-8, 15, and 16 in Table 1) are prepared by incubating aminated particles with 10 μg of DyLight 633 NHS ester for 2 hours at room temperature prior to antigen adsorption, cross-linking, or encapsulation; antigens in the remaining formulations (9-14 in Table 1) will be labeled with DyLight 633 NHS ester according to manufacturer's instructions. C57B1/6 BMDC is isolated and grown in culture with GM-CSF and IL-4 as previously described;³⁶ BMDCs prepared in this way are >90% CD11c⁺, immature, and highly endocytic. To assess BMDC uptake of the antigen formulations described in Table 1, incubate BMDCs with 10 μg of antigen or a corresponding quantity (˜30 μg) of empty MANPs or MSNPs for 30 minutes to 2 hours at 37° C. Quantify the percentage of BMDCs that are double-positive for CD11c and DyLight 633 by flow cytometry, similar to our assay for assessing uptake of bacterial antigens.³⁶ Confocal fluorescence microscopy experiments conducted at 4° C. and 37° C. are used to confirm internalization and to assess the kinetics of intracellular antigen release, which will be achieved via particle dissolution in the case of SLB-encapsulated MANPs and reduction of disulfide-containing crosslinkers in the endolysosomal environment in the case of MANPs with surface-oriented antigen. If the DEC-205 scFv promotes insufficient uptake by BMDCs, use SLBs modified with C3b and/or mannosylated cholesterol as described herein.

Since traditional alum adjuvants have been postulated to have a toxic effect on APCs,³⁷ monitor BMDC viability in each phagocytosis assay by including 7-aminoactinomycin D (7-AAD) to detect dead cells via flow cytometry. Assess the effect of MANPs and Imject® Alum on BMDC apoptosis over a period of 72 hours. To do so, BMDCs are incubated with the antigen formulations listed in Table 1 for two hours at 37° C., washed to remove extracellular antigen, and stained with FITC-labeled annexin V and propidium iodide (PI) immediately, as well as 12, 24, 48, and 72 hours post-exposure to assess cellular lysis and apoptosis.³⁸ Staining with FITC-labeled annexin V alone is indicative of the early stages of apoptosis, while staining with PI indicates a loss of membrane integrity associated with either the late stages of apoptosis or necrosis. Loss of viability immediately following MANP or Imject® Alum uptake will indicate that the formulation has a toxic effect. Apoptosis that occurs after the 24-hour time point may be due to toxicity of the antigen formulation or attributable to BMDC maturation and cytokine release,³⁸ both of which will be further evaluated as described herein.

### Example 11

### Quantify Expression of Co-Stimulatory Markers and Cytokine Production in BMDCs after Exposure to Antigen-Loaded MANPs

To measure the responses of BMDCs to MANP and Imject Alum formulations, incubate BMDCs with the antigen formulations listed in Table 1 for two hours at 37° C. and wash them to remove extracellular antigen. BMDC maturation is assessed after 24, 48, and 72 hours by staining for expression of surface markers (CD11c, CD40, CD83, CD80, CD86, MHC Class I and Class II, and CCR7), which is analyzed by flow cytometry.³⁶ Antibodies against an Iglc-derived peptide complexed with MHC class I molecules are developed; assess cross-presentation of Ig1C-derived peptides upon incubation of BMDCs with antigen formulations containing Ig1C. Supernatants collected from antigen-pulsed BMDC cultures are analyzed for cytokines (IL-1β, IL-6, TNF-α, IL-10, IL-12p70, IL-12p40, IFN-β, and IFN-γ) using a multiplex assay; these results will provide information about the type of DC activation induced by the different antigen formulations, as well as the types of T-cell responses that will be favored.

### Example 12

### Characterization of Proliferation of Naive CD8T Cells upon Incubation with MANP-Pulsed BMDCs

To evaluate the ability of MANPs to induce BMDCs capable of activating CD8T cells, first isolate CD8T cells from the lymph nodes of naïve C57B1/6 mice using MACS® MicroBeads (Miltenyi Biotec) to negatively-select unwanted cells. CD8T cells will then be incubated with BMDCs that have been pulsed with the antigen formulations in Table 1; preparation of antigen-pulsed BMDCs is optimized based on expression of maturation markers (subtask 4.4.2). Proliferative responses of CD8T cells is measured using a 5-(6)-carboxyfluorescein diacetate succinimidyl diester (CFSE) dilution assay. Antigen-specific CD8T-cell responses are evaluated in subtask 4.5.2 by comparing T-cell responses in mice immunized with antigen-loaded MANPs to T-cell responses in mice immunized with empty MANPs. The experiments of this example thus provide an in vitro assessment of APC uptake, maturation, and cytokine release, as well as CD8T cell activation and proliferation induced by the 8 antigen-loaded MANP formulations in comparison to traditional alum, which are used as predictors of immuneity.

### Example 13

### Quantification of Antigen-Specific Antibody and CD8T Cell Responses in Immunized C57B1/6 Mice

Quantify Antigen-Specific Antibody Titers as a Function of Time after Immunization with Antigen-Loaded MANPs. Assess antibody responses by vaccinating groups of five C57B1/6 mice intramuscularly with 10 μg of the antigen formulations listed in Table 1 on days 0 and 14; corresponding concentrations (˜30 μg) of empty MANPs and MSNPs will be used as negative controls. In order to follow the kinetics of the antibody response, sera is collected prior to the first immunization and on days 7, 14, 21, and 28. Serum-associated, antigen-specific IgG antibody titers are determined by end-point dilution ELISA, using Ig1C or LPS as target antigens.

Assess Time-Dependent T-Cell Responses after Immunization with Antigen-Loaded MANPs.

To assess the effect of MANP adjuvants on the activation of CD4⁺ and CD8⁺ T cells, immunize groups of twelve C57B1/6 mice intramuscularly with 10 μg of the antigen formulations listed in Table 1 on days 0 and 14; corresponding concentrations (˜30 μg) of empty MANPs and MSNPs are used as negative controls. Since T-cell responses begin as early as two days post-challenge and peak between four and seven days post-challenge,³⁹ collect draining lymph nodes on days 3, 7, 17, and 21 and assess the number of activated CD4⁺ and CD8⁺ T cells using flow cytometry. Specifically, quantify expression levels of CD44 and CD62L since naïve T cells typically express low levels of CD44 and high levels of CD62L, while activated T cells typically express high levels of CD44 and low levels of CD62L.⁴⁰ Determine whether T cell responses are antigen-specific by comparing responses in mice immunized with antigen-loaded MANPs to responses in mice immunized with empty MANPs.

Use OTI transgenic CD8T cells specific for the SIINFEKL peptide derived from ovalbumin⁴¹ to assess the level of memory CD8T-cell responses elicited by our MANP adjuvant platform, since corresponding reagents are not available for Ft-derived antigens. Adoptively transfer 1×10⁶ OTI Tg CD8T cells into three recipient mice and immunize with 10 μg of OVA-loaded MANPs on days 0 and 14; on day 42, use flow cytometry to quantify the percent of CD8T cells positive for the OTI transgenic T cell receptor (TCR). This experiment will demonstrate the effectiveness of MANPs in generating long-lasting CD8T-cell responses.

Identify Cells in the Draining Lymph Nodes that Internalize Antigen-Loaded MANPs.

To confirm successful transportation to draining lymph nodes and to determine the type(s) of APCs that internalize each of the antigen formulations listed in Table 1, immunize groups of four C57B1/6 mice with DyLight 633-labeled MANPs (formulations 1-8, 15, and 16) or antigens (formulations 9-14), prepared according to the description herein. Remove draining lymph nodes at 4, 12, 24, and 48-hours post-immunization and quantify cell populations positive for DyLight 633 and various APC markers, including but not limited to CD11c (DCs), CD11c/B220 (plasmacytoid DCs), CD11b or F4/80 (macrophages), and CD19 or B220 (B cells). The experiments of this example will provide an in vivo assessment of antigen-specific antibody and T-cell responses induced by the 8 antigen-loaded MANP formulations in comparison to traditional alum, as well as information about the types of draining lymph node APCs that internalize MANPs after intramuscular administration. These data will be used to demonstrate the adjuvant activity of the MANP platform.

### Example 14

### Challenge of Vaccinated C57B1/6 Mice with the Ft Subsp. Tularensis SCHU S4 Strain

Evaluate the adjuvant activity of antigen-loaded MANPs in comparison to conventional adjuvant formulations in a C57B1/6 mouse model of respiratory tularemia. To do so, mice are immunized following a prime-boost immunization regimen optimized to induce cellular and humoral immune responses. Vaccinated mice are then be challenged with escalating doses of the virulent F. tularensis SCHU S4 strain and monitored for improved protection Since vaccination/challenge studies with Ft LVS, which is currently used to immunize at-risk military personnel under the Special Immunization Program, suggest that vaccinated mice remain relatively susceptible to SCHU S4 challenge, perform additional studies in Fischer 344 rats, which develop much stronger immunity after vaccination.⁴²

### Example 15

### In-Depth Characterization of MANPs Loaded with Antigens Derived from Additional Bacteria, Viruses, or Toxins

MANPs are loaded with anthrax PA, Ebola sGP, Lassa sGP1, RTA, and/or BoNT-A LC and are tested as described in the experiments of the preceding examples. Challenge experiments with B. anthracis and Ebola virus may also be conducted using techniques that are described herein or that are well-known to those of ordinary skill in the art.

### Example 16

### Use of MANPs as an Adjuvant for Whole Bacteria or Viruses

The aerosol-assisted EISA process enables the generation of mesoporous oxide particles ranging in size from 30-nm to >10-μm. Therefore, the techniques that are described herein enable the synthesis of particles >100-nm in diameter with pores large enough (see FIGS. 1E and 1F) to accommodate inactivated or attenuated bacteria or viruses, such as the formalin-inactivated Venezuelan equine encephalitis virus vaccine strain TC-83, which is ˜60-nm in diameter.⁴³

### REFERENCES

1. Schmidt C S, et al. Expert Rev iew of Vaccines 6: 391-400 (2007).
2. Hornung V, et al. Nat Immunol 9: 847-56 (2008).
3. Li H, et al. The Journal of Immunology 181: 17-21 (2008).
4. Goto N, et al. Vaccine 11: 914-8 (1993).
5. Moon J J, et al. Nat Mater 10: 243-51 (2011).
6. Lu Y F, et al. Nature 398: 223-6 (1999).
7. Epler K, et al. Advanced Healthcare Materials 1: 241-(COVER) (2012).
8. Kirby C, et al. Nat Biotech 2: 979-84 (1984).
9. Mundargi R C, et al. Journal of Controlled Release 125: 193-209 (2008).
10. des Rieux A, et al. Journal of Controlled Release 116: 1-27 (2006).
11. Ashley C E, et al. Nat Mater 10: 389-97 (COVER) (2011).
12. Lee C-H, et al. Nano Today 4: 165-79 (2009).
13. Bachmann M F, et al. Nat Rev Immunol 10: 787-96 (2010).
14. Moon J J, et al. Advanced Materials 24: 3724-46 (2012).
15. Sandor N, et al. Immunobiology (2012).
16. Kawakami S, et al. Gene therapy 7: 292-9 (2000).
17. Dobrovolskaia M, et al. 2011. Analysis of Nanoparticle Effects on Maturation of Monocyte Derived Dendritic Cells In Vitro, Nanotechnology Characterization Laboratory, National Cancer Institure-Frederick
18. Davies J Q, et al. Isolation and Culture of Human Macrophages. pp. 105-16(2004).
19. Nguyen D N, et al. Proceedings of the National Academy of Sciences 109: E797-E803 (2012).
20. Moore N M, et al. The Journal of Gene Medicine 10: 1134-49 (2008).
21. Ashley C E, et al. ACS Nano 6: 2174-88 (COVER) (2012).
22. Kim J H, et al. Journal of the Ceramic Society of Japan 118: 805-9 (2010).
23. Cejka J. Applied Catalysis A: General 254: 327-38 (2003).
24. Negrete O A, et al. Journal of Virology 81: 10804-14 (2007).
25. Konduru K et al. Vaccine 29: 2968-77 (2011).
26. Illick M, et al. Virology Journal 5: 161 (2008).
27. Jia Q, et al. Vaccine 27: 1216-29 (2009).
28. Conlan J W, et al. Vaccine 20: 3465-71 (2002).
29. Lu Z, et al. Immunology Letters 112: 92-103 (2007).
30. Savitt A G, et al. Clinical and Vaccine Immunology 16: 414-22 (2009).
31. Mara-Koosham G, et al. Infection and Immunity 79: 1770-8 (2011).
32. Ivins B, et al. Vaccine 13: 1779-84 (1995).
33. Ravichandran E, et al. Infection and Immunity 75: 3043-54 (2007).
34. Vitetta E S, et al. Proceedings of the National Academy of Sciences of the United States of America 103: 2268-73 (2006).
35. Johnson T S, et al. Clinical Cancer Research 14: 8169-77 (2008).
36. Thomas-Rudolph D, et al. The Journal of Immunology 178: 7283-91 (2007).
37. Goto N, et al. Vaccine 15: 1364-71 (1997).
38. Colino J, et al. The Journal of Immunology 171: 2354-65 (2003).
39. Moore T V, et al. American Journal of Respiratory Cell and Molecular Biology 45: 843-50 (2011).
40. Roberts A D, et al. The Journal of Immunology 172: 6533-7 (2004).
41. Clarke S R, et al. Immunol Cell Biol 78: 110-7 (2000).
42. Wu T H, et al. Vaccine 27: 4684-93 (2009).
43. Cole F E, et al. Applied Microbiology 27: 150-3 (1974).

What is claimed is:

1. A mesoporous alum nanoparticle comprising an antigen loaded into, absorbed or crosslinked to said nanoparticle.

2. The mesoporous alum nanoparticle according to claim 1 wherein said antigen is crosslinked to said nanoparticle to facilitate an immune response to said antigen in a subject or patient.

3. The mesoporous alum nanoparticle according to claim 1 wherein said antigen is loaded into said nanoparticle, said nanoparticle comprising a supported lipid bilayer, optionally modified with a ligand that facilitates uptake of the nanoparticles by antigen-presenting cells (APCs) and cytosolic disperson of antigen.

4. A mesoporous alum nanoparticle according to any of claims 1-3 comprising at least one physicochemically disparate antigen and optionally, at least one immunogenic molecule.

5. The mesoporous alum nanoparticle according to any of claims 1-4 wherein said antigens are loaded into said nanoparticle and crosslinked to the surface of said nanoparticle.

6. The mesoporous alum nanoparticle according to any of claims 1-5 comprising a supported lipid bilayer, at least one antigen, optionally at least one immunogenic molecule and at least one further component selected from the group consisting of:
a cell targeting species;
a ligand that facilitates uptake of the nanoparticles by antigen-presenting cells (APCs) and/or cytosolic dispersion of antigen;
a fusogenic peptide that promotes endosomal escape of nanoparticles and encapsulated DNA, and/or other cargo comprising at least one additional cargo component (other than the antigen) selected from the group consisting of polynucleotides (DNA or RNA), including double stranded linear DNA, minicircle DNA or a plasmid DNA;
at least one drug;
an imaging agent,
small interfering RNA, small hairpin RNA, microRNA, immunostimulatory RNA or a mixture thereof,
wherein one of said cargo components is optionally conjugated further with a nuclear localization sequence.

7. The mesoporous alum nanoparticle according to any of claims 4-6 wherein said immunostimulatory molecule is at least one molecule selected from the group consisting of a cytokine or a molecule selected from the group consisting of andrographolide, 14-deoxyandrographolide and 14-deoxy-11,12-didehydroandrographolide.

8. The mesporous alum nanoparticle according to any of claims 4-7 wherein said cytokine is at least one molecule selected from the group consisting of an interleukin, an interferon, GM-CSF and a tumor necrosis factor.

9. A pharmaceutical composition comprising an effective population of nanoparticles according to any of claims 1-8 in combination with a pharmaceutically acceptable carrier, additive or excipient, optionally in combination with an additional bioactive agent.

10. The composition according to claim 9 wherein said additional bioactive agent is an antibiotic or an antiviral agent.

11. The composition according to claim 9 wherein said additional bioactive agent is a cytokine, a molecule selected from the group consisting of andrographolide, 14-deoxyandrographolide and 14-deoxy-11,12-didehydroandrographolide or a mixture thereof.

12. The composition according to claim 9 or 11 wherein said cytokine is an interleukin, an interferon, GM-CSF, a tumor necrosis factor or a mixture thereof.

13. A method of eliciting an immune response in a subject or patient comprising administering to said subject or patient an effective amount of a composition according to any of claims 9-12.

14. A method of reducing the likelihood that a subject or patient will contract a disease state or condition from an infectious agent, said method comprising administering to said subject or patient an effective amount of a composition according to any of claims 9-12 comprising an antigen from said infectious agent to which said subject or patient elicits an immune response, said immune response reducing the likelihood that said infectious agent will cause said disease state or condition in said patient or subject.

15. A nanoparticle comprising:
a porous nanoparticle core; and
a cargo comprising at least one of an adjuvant, a protein antigen and a non-protein immunostimulant disposed with the particle and optionally, a lipid bilayer coating said core.

16. The protocell of claim 15, wherein the nanoparticle comprises an aluminum salt.

17. The protocell of claim 16, wherein the aluminum salt is alum boehmite and/or gibbsite.

18. The protocell of any of claims 15-17, wherein the cargo comprises protein antigens.

19. The protocell of any of claims 15-18, wherein the antigens are adsorbed to the core.

20. The protocell of claim 15-19, wherein the at least a portion of the antigens are cross-linked.

21. The protocell of any of claims 15-20, further comprising a lipid bilayer coating the core.

22. The protocell of any of claims 15-21, wherein the lipid bilayer is modified with ligands that promote uptake of antigen-presenting cells.

23. A method comprising:
loading a porous nanoparticle with a cargo comprising one of an adjuvant, an antigen and a non-protein immunostimulant and optionally, coating said loaded nanoparticle with a lipid bilayer.

24. The method of claim 23, wherein the particle is further coated with at least one lipid bilayer.

25. The method of claim 23 or 24, wherein loading comprises submersing the particle in a solution comprising the cargo.

26. The method of any of claims 23-25, further comprising modifying the lipid bilayer with ligands that promote uptake of antigen presenting cells.

27. The method of any of claims 23-26, wherein the nanoparticle comprises an aluminum salt and the cargo comprises protein antigens.

28. The method of any of claims 23-27, wherein the antigens are adsorbed to the particle.

29. The method of any of claims 23-28, further comprising cross-linking a portion of the antigens.

30. A method comprising:
administering a protocell comprising a porous nanoparticle and a cargo comprising one of an adjuvant, an antigen and a non-protein immunostimulant with the particle.

31. The method of claim 30, wherein the nanoparticle comprises an aluminum salt and the cargo comprises at least one protein antigen.

32. A mesoporous alum nanoparticle which has a pore size of approximately 0.03 nm to approximately 75 nm (preferably about 1 nm to about 75 nm) and which is loaded with:
(a) one or more antigens selected from the group consisting of a glycoprotein or lipoprotein derived from a Category A or B biothreat bacteria, virus or toxin; and, optionally
(b) a targeting ligand and at least one agent selected from the group consisting of a therapeutic small molecule, a siRNA, a shRNA, immunostimulatory RNA (isRNA) and/or a packaged plasmid DNA.

33. The mesoporous alum nanoparticle of claim 32, wherein the antigen is a glycoprotein or lipoprotein derived from one or more of the following: E. coli O157117 lipopolysaccharide (LPS), anthrax protective antigen (PA), soluble Nipah virus glycoprotein (sG), ricin toxin A-chain (RTA), ovalbumin (OVA), F. tularensis lipopolysaccharide, recombinant Bacillus anthracis protective antigen, recombinant botulinum neurotoxin type A (BoNT-A) light chain (LC), Zaire Ebola virus glycoprotein (sGP), filo- and arenavirus antigens, Ig1C, PA, sGP, sGP1, RTA, and BoNT-A LC, formalin-inactivated Venezuelan equine encephalitis virus vaccine strain TC-83 and lysozyme (LSZ).

34. The mesoporous alum nanoparticle of claim 32 or 33, wherein the nanoparticle is modified with one or more amine-containing silanes selected from the group consisting of (3-aminopropyl)triethoxysilane (APTES), (3-aminopropyl)-diethoxy-methylsilane (APDEMS) and (3-aminopropyl)-dimethyl-ethoxysilane (APDMES) and (3-aminopropyl)-trimethoxysilane (APTMS) to positively charge its pores and optionally, with hexamethyldisilazane (HMDS) to increase the hydrophobicity of its pores.

35. The mesoporous alum nanoparticle of any of claims 32-34, wherein the nanoparticle is encapsulated within a supported lipid bi-layer comprised of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof.

36. The mesoporous alum nanoparticle of claim 35, wherein the supported lipid bi-layer is modified with one or more compositions selected from the group consisting of human IgG, human complement (C3), mannosylated cholesterol and the TLR-4 agonist, monophosphoryl lipid A (MPLA).

37. The mesoporous alum nanoparticle of any of claims 32-36, wherein the nanoparticle retains its antigen under neutral or basic pH conditions and releases antigen under acidic pH conditions.

38. The mesoporous alum nanoparticle of any of claims 32-37, wherein the nanoparticle further comprises an immunostimulatory RNA (isRNA).

39. The mesoporous alum nanoparticle of any of claims 32-38, wherein the antigen comprises about 50% to about 70% by weight of the nanoparticle.

40. The mesoporous alum nanoparticle of any of claims 32-39, wherein the antigen comprises about 20% to about 40% by weight of the nanoparticle.

41. The mesoporous alum nanoparticle of any of claims 35-40, wherein the supported lipid bi-layer comprises an endosomolytic peptide.

42. The mesoporous alum nanoparticle of any of claims 32-41, wherein the antigen is amphiphilic and the nanoparticle is modified with hexamethyldisilazane (HMDS).

43. The mesoporous nanoparticle of any of claims 32-42, wherein the antigen is cross-linked by modifying the nanoparticle with one or more compositions selected from the group consisting of (3-mercaptopropyl)trimethoxysilane (MPTS), sulfosuccinimidyl-4-(N-maleimidomethyl)cyclohexane-1-carboxylate (sulfo-SMCC) and sulfosuccinimidyl 6-(3′-[2-pyridyldithio]-propionamido)hexanoate (sulfo-LC-SPDP).

44. The mesoporous alum nanoparticle of any of claims 32-43, wherein the nanoparticle is modified with one or more amine-containing silanes selected from the group consisting of (3-aminopropyl)triethoxysilane (APTES), (3-aminopropyl)-diethoxy-methylsilane (APDEMS) and (3-aminopropyl)-dimethyl-ethoxysilane (APDMES) and (3-aminopropyl)-trimethoxysilane (APTMS).

45. The mesoporous alum nanoparticle of any of claims 32-44, wherein the nanoparticle is a mesoporous aluminum hydroxide or a mesoporous aluminum sulfate nanoparticle.

46. The mesoporous alum nanoparticle of any of claims 32-45, wherein the mesoporous alum nanoparticle is made by aerosol-assisted evaporation-induced self-assembly.

47. The mesoporous alum nanoparticle of any of claims 32-46, wherein the nanoparticle has a pore size of approximately 1 nm to approximately 75 nm, a surface area of approximately 75 m²/g to approximately 1,500 m²/g and a diameter of approximately about 50 nm to about 50 μm.

48. A pharmaceutical composition comprising a plurality of mesoporous nanoparticles of any of claims 32-47 and, optionally, one or more pharmaceutically acceptable excipients.

49. Use of a composition according to any of claims 9-12 and 47 in the manufacture of a medicament for inducing an immune response in a patient or subject.

50. Use of a composition according to any of claims 9-12 and 47 in the manufacture of a medicament for reducing the likelihood that a subject or patient will contract a disease state or condition from an infectious agent.
